(19) United States
(12) Patent Application Publication
(10) Pub. No.: US 2021/0100246 A1
(43) Pub. Date: Apr. 8, 2021

(54) PYRIDYL-FORMAMIDINES HAVING A FUNGICIDAL ACTIVITY, THEIR AGRONOMIC COMPOSITIONS AND USE THEREOF

(71) Applicant: ISAGRO, S.p.A., Milan (IT)

(72) Inventors: Entela SINANI, Novara (IT); Daniele FORGIA, Borgomanero (NO) (IT); Marilena GUSMEROLI, Monza (MB) (IT); Paolo BELLANDI, Carcare (SV) (IT); Matteo VAZZOLA, Cogliate (MB) (IT); Christian BADARACCO, Vittuone (IT); Riccardo LIGUORI, Monza (MB) (IT)

(21) Appl. No.: 16/496,802

(22) PCT Filed: Mar. 26, 2018
(86) PCT No.: PCT/IB2018/052051

§ 371 Date: Sep. 23, 2019

(30) Foreign Application Priority Data
Mar. 27, 2017 (IT) 102017000033543

(51) Int. Cl. A01N 43/40; C07D 213/74; C07D 417/12; A01N 43/82; A01N 43/78; C07D 413/12; A01N 43/80; C07D 401/12

(52) U.S. Cl. CPC A01N 43/40; C07D 213/74; C07D 417/12; C07D 401/12; A01N 43/78; C07D 413/12; A01N 43/80; A01N 43/82

(57) ABSTRACT

New pyridyl-formamidines having general formula (I): are described, together with agronomic compositions comprising said compounds having formula (I) and at least one other active ingredient compatible therewith, selected from fungicides different from those having general formula (I), and their relative use for the control of phytopathogenic fungi of agricultural crops.

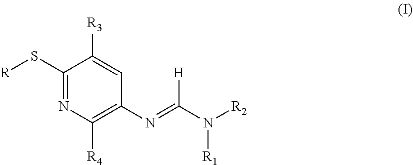

The present invention relates to pyridyl-formamidines having a high fungicidal activity; in particular, it relates to thiopyridyl-formamidines suitably substituted, having a high fungicidal activity and their use for the control of phytopathogenic fungi of important agricultural crops.

Pyridyl-formamidines having a high fungicidal activity are already known and are described, in particular, in patent applications EP2264011, EP2264012, WO2008/101682, WO2012/146125 and WO2015/155075.

The products described in these documents, however, are often unsatisfactory both from the point of view of the level of activity against phytopathogenic fungi and, or alternatively, from the point of view of phytotoxicity with respect to important agricultural crops.

The Applicant has now surprisingly found that new thiopyridyl-formamidines characterized by a pyridine bearing the nitrogen atom in a meta position with respect to the formamidine residue and by the presence of alkyl- or arylthio-groups in position 2 of the pyridine ring, in addition to exhibiting an excellent fungicidal activity at low doses, are very well tolerated by many plant species, thus allowing the practical use of these compounds for the control of phytopathogenic microorganisms of important agricultural crops.

The object of the present invention therefore relates to pyridyl-formamidines having general formula (I):

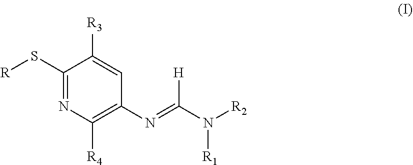

wherein:
R represents a hydrogen; a C₁-C₁₂ alkyl; a C₁-C₁₂ haloalkyl; a C₂-C₁₂ alkenyl; a C₂-C₁₂ haloalkenyl; a C₂-C₁₂ alkinyl; a C₂-C₁₂ haloalkinyl; a C₃-C₁₄ cycloalkyl; a C₄-C₁₈ cycloalkylalkyl; a C₃-C₁₄ cycloalkenyl; a C₃-C₁₄ halocycloalkyl; a C₄-C₁₈ cycloalkenylalkyl; a formyl; a C₂-C₁₂ alkylcarbonyl; a C₂-C₁₂ haloalkylcarbonyl; a C₃-C₁₂ alkenylcarbonyl; a C₄-C₁₄ cycloalkylcarbonyl; or
R represents a C₁-C₆-alkyl-B—C₁-C₁₂-alkyl; C₁-C₆-haloalkyl-B—C₁-C₁₂-alkyl; C₁-C₆-alkyl-B—C₁-C₁₂-haloalkyl; C₁-C₆-haloalkyl-B—C₁-C₁₂-haloalkyl; C₃-C₈-C₃-C₁₄-cyclo-alkyl-B—C₁-C₁₂-alkyl; C₃-C₁₄-cycloalkyl-B—C₁-C₁₂-halo-alkyl; C₁-C₆-alkyl-B—C₃-C₁₄-cycloalkyl; C₁-C₆-alkyl-B—C₃-C₁₄-halocycloalkyl; C₃-C₁₄-cyclo-alkyl-B—C₃-C₁₄-cyclo-alkyl; C₄-C₁₈-cycloalkylalkyl-B—C₃-C₁₄-cycloalkyl; C₁-C₆-alkyl-B—C₂-C₁₂-alkenyl; or
R represents A-; A-(C₁-C₆ alkyl)-; A-(C₁-C₆ haloalkyl)-; A-(C₃-C₁₄ cycloalkyl)-; A-(C═O)—; A-(C₁-C₆ alkyl)-(C═O)—; A-B—(C₁-C₁₂ alkyl)-; A-B—(C₁-C₁₂ haloalkyl); A-B—(C₃-C₁₄ cycloalkyl)-; A-(C₁-C₁₂ alkyl)-B—(C₁-C₁₂ alkyl); A-(C₁-C₁₂ alkyl)-B—(C₁-C₁₂-haloalkyl); A-B-A-; (C₁-C₆ alkyl)-B-A-; (C₁-C₆ haloalkyl)-B-A-; (C₃-C₁₄ cycloalkyl)-B-A-; A-B-A-(C₁-C₆ alkyl)-; A-B-A-(C₃-C₁₄ cycloalkyl)-; (C₁-C₆ alkyl)-B-A-(C₁-C₆ alkyl)-; (C₃-C₁₄ cycloalkyl)-B-A-(C₁-C₆ alkyl)-; (C₁-C₆ haloalkyl)-B-A-(C₁-C₆ alkyl)-;
A represents an aromatic mono- or bicyclic carbocyclic group possibly substituted by one or more groups, the same or different, preferably selected from halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl; or a condensed monocycle or bicycle with 3-12 terminals, possibly aromatic, partially or completely saturated and which contains from 1 to 4 heteroatoms selected from nitrogen, oxygen and sulfur, with the proviso that these cyclic systems with 3-12 terminals do not contain —O—O—, —S—S—, —O—S— fragments, said cyclic systems with 3-12 terminals being possibly substituted by one or more groups, the same or different, preferably selected from halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl;
B represents —(C═O)—; —C(═NOR₅)—; O—(C═O)—; —C(═O)—O—; —O—; —S—; —N(R₆)—(C═O)—; or —(C═O)—N(R₆)—;
R₁ represents a C₁-C₆ alkyl,
R₂ represents a C₂-C₆ alkyl;
   or R₁ and R₂, jointly with the N atom to which they are bound, form a heterocyclic ring containing from 4 to 7 atoms, possibly substituted by halogen atoms;
R₃ and R₄, the same or different, represent a hydrogen atom; a halogen atom; a C₁-C₆ alkyl; a C₁-C₆ alkoxyl; a C₁-C₆ haloalkoxyl, a CF₃ group; a CF₂H group; a CFH₂ group; a cyano group;
R₅ and R₆ represent a hydrogen atom; a C₁-C₆ alkyl, a C₁-C₆ haloalkyl; a C₃-C₆ cycloalkyl; a benzyl or aryl group possibly substituted by one or more groups, the same or different, preferably selected from halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl;

with the proviso that when R₃ is a hydrogen atom, a halogen atom, a cyano group, a C₁-C₆ alkyl or a C₁-C₆ alkoxyl, R₄ is different from a hydrogen atom, a halogen atom or a cyano group.

Examples of halogen are fluorine, chlorine, bromine, iodine.

Examples of C₁-C₁₂ alkyl are: methyl, ethyl, n-propyl, isopropyl, n-butyl, isobutyl, sec-butyl, tert-butyl, n-pentyl, 3-methylbutyl, n-hexyl, 3,3-dimethyl-butyl.

Examples of C₁-C₁₂ haloalkyl are: fluoromethyl, difluoromethyl, trifluoromethyl, chloromethyl, dichloromethyl, 2,2,2-trifluoroethyl, 1,1,2,2-tetrafluoroethyl, pentafluoroethyl, heptafluoropropyl, 4,4,4-trichlorobutyl, 4,4-difluoropentyl, 5,5-difluorohexyl.

Examples of C₃-C₁₄ cycloalkyl are: cyclopropyl, cyclobutyl, cyclopentyl, cyclohexyl.

Examples of C₃-C₁₄ halocycloalkyl are: 2,2-dichloro-cyclopropyl, 2,2-difluorocyclopropyl, 2,2,3,3-tetrafluorocyclobutyl, 3,3-difluorocyclopentyl, 2-fluorocyclohexyl.

Examples of C₂-C₁₂ alkenyl are: ethenyl, propenyl, butenyl.

Examples of C₂-C₁₂ haloalkenyl are: 2,2-dichloro-propenyl, 1,2,2-trichloropropenyl.

Examples of C₂-C₁₂ alkinyl are: ethinyl, propargyl.

An example of a C₂-C₁₂ haloalkinyl is 3-chloropropinyl.

Examples of C₃-C₁₄ cycloalkyl are: cyclopropyl, cyclobutyl, cyclopentyl, cyclohexyl.

Examples of C₃-C₁₄ halocycloalkyl are: 2,2-dichloro-cyclopropyl, 2,2-difluorocyclopropyl, 2,2,3,3-tetrafluorocyclobutyl, 3,3-difluorocyclopentyl, 2-fluorocyclohexyl.

Examples of C₃-C₁₄ cycloalkenyl are: cyclobutyl, cyclopentenyl, cyclohexenyl.

Examples of C₄-C₁₈ cycloalkylalkyl are: 2-ethylcyclopropyl, cyclopentylmethyl, 3-propylhexyl.

Examples of C₃-C₁₄ cycloalkenyl are: cyclopropene, cyclohexene, cyclopentene.

Examples of C₂-C₁₂ alkylcarbonyl are: methylcarbonyl, ethylcarbonyl, isopropylcarbonyl, butylcarbonyl.

Examples of C₃-C₁₂ alkenylcarbonyl are: 2-propenylcarbonyl, 2-butenylcarbonyl, 3-pentenyl-carbonyl.

Examples of C₂-C₁₂ haloalkylcarbonyl are: fluoromethylcarbonyl, difluoromethylcarbonyl, trifluoromethylcarbonyl, dichloromethylcarbonyl, 2,2,2-trifluoroethylcarbonyl.

Examples of C₄-C₁₄ cycloalkylcarbonyl are: cyclopropylcarbonyl, cyclopentylcarbonyl, cyclohexyl-carbonyl.

Examples of C₁-C₆ alkoxyl are: methoxyl, ethoxyl.

Examples of C₁-C₆ haloalkoxyl are: trifluoromethoxyl, 1,1,2,2-tetrafluoroethoxyl, 1,1,2,3,3,3-hexafluoro-propyloxyl.

Examples of C₄-C₁₅ cycloalkoxyl are: cyclopropoxyl, cyclopentoxyl.

Examples of heterocyclic rings having from 4 to 7 atoms, possibly halogenated, are: azetidine, 3,3-difluoroazetidine. pyrrolidine, piperidine, 4-fluoropiperidine. morpholine.

The following also fall within the spirit of the present invention:

a) all possible geometric isomers of the compounds having general formula (I) deriving from particular meanings of the substituents R—R₄;

b) the salts of the compounds having general formula (I) obtained by the addition of inorganic or organic acids.

An object of the present invention therefore also relates to pyridyl-formamidines that are a) mixtures of non-separated geometric isomers, mixtures of partially separated geometric isomers, single geometric isomers; b) in the form of salts obtained by the addition of inorganic or organic acids.

Examples of preferred compounds having general formula (I) are compounds wherein R, R₁, R₂, R₃ and R₄ have the meanings indicated in Table 1:

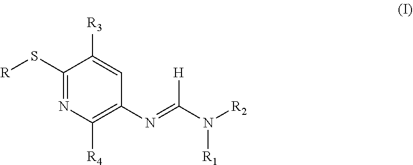

TABLE 1

| Compound | | | | | |
|---|---|---|---|---|---|
| Nr. | R | R1 | R2 | R3 | R4 |
| 1. | 3-CF₃-benzyl | CH₃ | Et | Br | CH₃ |
| 2. | 3-methyl-1-butyl | CH₃ | Et | CH₃ | CH₃ |
| 3. | 3-CF₃-benzyl | CH₃ | Et | CH₃ | CH₃ |
| 4. | 3-CH₃-benzyl | CH₃ | Et | CH₃ | CH₃ |
| 5. | 3-methyl-1-butyl | CH₃ | Et | Br | CH₃ |
| 6. | 3-CF₃-phenyl | CH₃ | Et | Br | CH₃ |
| 7. | 3-CF₃-phenyl | CH₃ | Et | H | CH₃ |
| 8. | 4-Cl-3-CF₃-phenyl | CH₃ | Et | Br | CH₃ |
| 9. | 3-CF₃-phenyl | CH₃ | Et | CH₃ | CH₃ |
| 10. | 3-propoxy-2-propyl | CH₃ | Et | Br | CH₃ |
| 11. | 3-methyl-1-butyl | CH₃ | Et | H | CH₃ |
| 12. | 3-propoxy-2-propyl | CH₃ | Et | CH₃ | CH₃ |
| 13. | 5-CH₃-1,3,4-thiadiazol-2-yl | CH₃ | Et | Br | CH₃ |
| 14. | 3-CF₃-phenyl | CH₃ | Et | Br | OMe |
| 15. | 3,4,4-trifluorobut-3-en-1-yl | CH₃ | Et | CH₃ | CH₃ |
| 16. | benzyl | CH₃ | Et | CH₃ | CH₃ |
| 17. | n-hexyl | CH₃ | Et | CH₃ | CH₃ |
| 18. | 4-Cl-3-CF₃-benzyl | CH₃ | Et | CH₃ | CH₃ |
| 19. | 2-fluoro-benzyl | CH₃ | Et | CH₃ | CH₃ |
| 20. | 3-methyl-thiazol-2-yl | CH₃ | Et | CH₃ | CH₃ |
| 21. | 2-ethyl-1-hexyl | CH₃ | Et | CH₃ | CH₃ |
| 22. | 3-CF₂H-benzyl | CH₃ | Et | CH₃ | CH₃ |
| 23. | n-decyl | CH₃ | Et | CH₃ | CH₃ |
| 24. | 1-adamantyl | CH₃ | Et | CH₃ | CH₃ |
| 25. | cyclododecyl | CH₃ | Et | Br | CH₃ |
| 26. | 5-Br-2-CH₃-thiazol-4-yl | CH₃ | Et | CH₃ | CH₃ |
| 27. | (3-tertbutyl-5-isoxazolyl)methyl | CH₃ | Et | CH₃ | CH₃ |
| 28. | 2-isopropoxy-carbonyl-1-ethyl | CH₃ | Et | CH₃ | CH₃ |
| 29. | 5,5-dimethyl-3-methoxyimino-1-pentyl | CH₃ | Et | Br | CH₃ |
| 30. | 2-(cyclopropyl-aminocarbonyl)-1-ethyl | CH₃ | Et | CH₃ | CH₃ |
| 31. | 4,4-dimethyl-3-oxa-1-pentyl | CH₃ | Et | Br | CH₃ |
| 32. | (2,2-dichloro-1-methyl-cyclopropyl)-methyl | CH₃ | Et | CH₃ | CH₃ |
| 33. | 3-(ethylthio)-1-butyl | CH₃ | Et | CH₃ | CH₃ |
| 34. | 3-[3-(2-oxa-propyl)-cyclohexyl]-1-propyl | CH₃ | Et | CH₃ | CH₃ |
| 35. | 5,5-dimethyl-3-isoxazolyl | CH₃ | Et | Br | CH₃ |
| 36. | (2-naphthyl)methyl | CH₃ | Et | CH₃ | CH₃ |
| 37. | 2-phenyl-1-ethyl | CH₃ | Et | CH₃ | CH₃ |
| 38. | cyclopropanoyl | CH₃ | Et | CH₃ | CH₃ |
| 39. | 2,6-difluorobenzyl | CH₃ | Et | CH₃ | CH₃ |
| 40. | Cyclohexylmethyl | CH₃ | Et | CH₃ | CH₃ |
| 41 | 3-CF₃-phenyl | CH₃ | Et | Br | Cl |
| 42 | 3-methyl-1-butyl | CH₃ | Et | Br | Br |
| 43 | 2-fluoro-3-CF₃-benzyl | CH₃ | Et | CH₃ | CH₃ |
| 44 | 3-CF₃-phenyl | CH₃ | Et | Br | Br |
| 45 | 4-t-butyl-benzyl | CH₃ | Et | CH₃ | CH₃ |
| 46 | 1-adamantyl-methyl | CH₃ | Et | CH₃ | CH₃ |
| 47 | 2-fluoro-4,6 diCF₃-benzyl | CH₃ | Et | CH₃ | CH₃ |
| 48 | 4-fluoro-benzyl | CH₃ | Et | CH₃ | CH₃ |
| 49 | 4-F-3-CF₃-benzyl | CH₃ | Et | CH₃ | CH₃ |
| 50 | 1-phenyl-ethyl | CH₃ | Et | CH₃ | CH₃ |
| 51 | 2-iodo-benzyl | CH₃ | Et | CH₃ | CH₃ |
| 52 | cyclohexyl | CH₃ | Et | CH₃ | CH₃ |
| 53 | 2-CH₃-benzyl | CH₃ | Et | CH₃ | CH₃ |
| 54 | 2-CF₃-benzyl | CH₃ | Et | CH₃ | CH₃ |
| 55 | 2-fluoro-6-CF₃-benzyl | CH₃ | Et | CH₃ | CH₃ |
| 56 | 2-chloro-benzyl | CH₃ | Et | CH₃ | CH₃ |
| 57 | 2-bromo-benzyl | CH₃ | Et | CH₃ | CH₃ |
| 58 | 2-(cyclohexyl)-ethyl | CH₃ | Et | CH₃ | CH₃ |
| 59 | cyclooctyl | CH₃ | Et | CH₃ | CH₃ |
| 60 | 2-Br-6-OCF₂H-benzyl | CH₃ | Et | CH₃ | CH₃ |
| 61 | 2-Br-6-CF₃-benzyl | CH₃ | Et | CH₃ | CH₃ |
| 62 | 2-OCF₂CF₂H-6-CF₃-benzyl | CH₃ | Et | CH₃ | CH₃ |
| 63 | Cyclopentylmethyl | CH₃ | Et | CH₃ | CH₃ |
| 64 | Cyclopentyl | CH₃ | Et | CH₃ | CH₃ |
| 65 | 1-methyl-3-CF₃-5-OCH₂CF₃-4-pyrazolylmethyl | CH₃ | Et | CH₃ | CH₃ |
| 66 | 4-pyridylmethyl | CH₃ | Et | CH₃ | CH₃ |
| 67 | 2-pyridylmethyl | CH₃ | Et | CH₃ | CH₃ |
| 68 | 5,5 dimethyl-2 isoxazolin-3-yl | CH₃ | Et | CH₃ | CH₃ |
| 69 | 5-CH₃-1,3,4-thiadiazol-2-yl | CH₃ | Et | CH₃ | CH₃ |
| 70 | cyclopropylmethyl | CH₃ | Et | CH₃ | CH₃ |
| 71 | 3-pyridylmethyl | CH₃ | Et | CH₃ | CH₃ |
| 72 | 2-methyl-4-CF₂H-5-thiazolylmethyl | CH₃ | Et | CH₃ | CH₃ |
| 73 | 1-methyl-3-CF₃-5-OCF₂H-4-pyrazolylmethyl | CH₃ | Et | CH₃ | CH₃ |
| 74 | 1-methyl-3-CF₃-5-OCH₂Si(CH₃)₃-4-pyrazolylmethyl | CH₃ | Et | CH₃ | CH₃ |
| 75 | 1-methyl-3-CF₂H-4-pyrazolylmethyl | CH₃ | Et | CH₃ | CH₃ |
| 76 | 2-methyl-4-CF₃-5-thiazolylmethyl | CH₃ | Et | CH₃ | CH₃ |
| 77 | 2-thienyl-2-ethyl | CH₃ | Et | CH₃ | CH₃ |
| 78 | 2-Cl-4,5 methylenedioxy-benzyl | CH₃ | Et | CH₃ | CH₃ |
| 79 | 3-Si(CH₃)₃-propyl | CH₃ | Et | CH₃ | CH₃ |
| 80 | n-hexyl | CH₃ | Et | Br | CH₃ |
| 81 | cyclohexylmethyl | CH₃ | Et | Br | CH₃ |
| 82 | 2-oxo-2-phenylethyl | CH₃ | Et | CH₃ | CH₃ |
| 83 | 3-CH(OCH₃)₂-(2-oxo-1,3-oxazolidin-5-yl)-methyl | CH₃ | Et | CH₃ | CH₃ |

Preferred compounds having formula (I) are those wherein:
R represents a C₁-C₁₂ alkyl, a C₁-C₁₂ haloalkyl, a C₂-C₁₂ haloalkenyl, a C₃-C₁₄ cycloalkyl, a C₄-C₁₈ cycloalkylalkyl, A-, A-(C₁-C₆ alkyl);
A represents an aromatic mono- or bicyclic carbocyclic group possibly substituted by one or more groups, the same or different, preferably selected from halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl; or a condensed monocycle or bicycle with 3-12 terminals, possibly aromatic, partially or completely saturated and which contains from 1 to 4 heteroatoms selected from nitrogen, oxygen and sulfur, with the proviso that these cyclic systems with 3-12 terminals do not contain —O—O—, —S—S—, —O—S— fragments, said cyclic systems with 3-12 terminals being substituted by one or more groups, the same or different, preferably selected from halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl;
R₁ represents a C₁-C₆ alkyl;
R₂ represents a C₂-C₆ alkyl;
R₃ and R₄ represent a halogen atom, a C₁-C₆ alkyl, with the proviso that when R₃ is a halogen atom, R₄ is not a halogen atom.

Compounds having formula (I) wherein R₃ and R₄ represent a C₁-C₆ alkyl, are even more preferred.

Compounds having formula (I) wherein R, R₁, R₂, R₃ and R₄ have the following meanings, are particularly preferred:

| Compound | | | | | |
|---|---|---|---|---|---|
| Nr. | R | R₁ | R₂ | R₃ | R₄ |
| 1. | 3-CF₃-benzyl | CH₃ | Et | Br | CH₃ |
| 2. | 3-methylbutyl | CH₃ | Et | CH₃ | CH₃ |
| 3. | 3-CF₃-benzyl | CH₃ | Et | CH₃ | CH₃ |
| 17. | n-hexyl | CH₃ | Et | CH₃ | CH₃ |
| 39. | 2,6-difluorobenzyl | CH₃ | Et | CH₃ | CH₃ |
| 40. | cyclohexylmethyl | CH₃ | Et | CH₃ | CH₃ |

The compounds having general formula (I) are prepared from the corresponding aniline having formula (II), according to the reaction scheme 1.

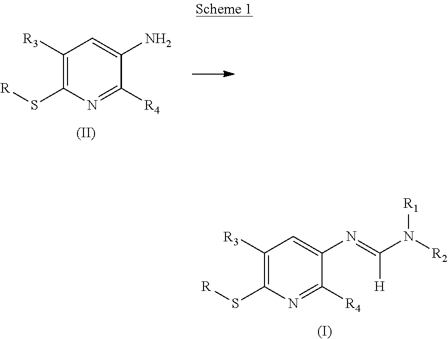

Various methods for effecting this transformation are known in literature; the most widely used are the following:

a) treatment of the aniline having formula (II) with an acetal having formula R₁R₂NC(OR₇), wherein R₇ represents an alkyl group, according to what is described in “Synthetic Communications”, 24 (1994), pages 1617-1624;

b) treatment of the aniline having formula (II) with an amide having formula HCONR₁R₂ in the presence of POCl₃ or SOCl₂, according to what is described in “Tetrahedron”, 46 (1990), pages 6058-6112;

c) treatment of the aniline having formula (II) with an orthoester having formula HC(OR₇), wherein R₇ represents an alkyl group, to form the corresponding imino-ether, followed by heating the same in the presence of an amine having formula HNR₁R₂, according to what is described in US4209319;

d) treatment of the aniline having formula (II) with phosgene to form the corresponding isocyanate followed by reaction with an amide having formula HCONR₁R₂, according to what is described in WO 00/46184;

e) treatment of the aniline having formula (II) with C₂H₅OCH═NCN to form a N-cyanoamidine, followed by reaction with an amine having formula HNR₁R₂, according to what is described in WO 00/46184;

f) treatment of the aniline having formula (II) with N,N-dimethylformamide in the presence of a sulfonyl-chloride, such as, for example, 2-pyridylsulfonylchloride or phenylsulfonylchloride, to form the corresponding di-methylamidine (R₁═R₂═Me) followed by reaction with an amine having formula HNR₁R₂, according to what is described in “Tetrahedron”, 56 (2000), pages 8253-8262 and in “Journal Combinatorial Chemistry” 11 (2009), pages 126-130.

The compound having formula (II) can be prepared by reduction of the corresponding nitroderivative having formula (III), as indicated in reaction scheme 2, according to methods well-known in organic chemistry, as described for example in “Advanced Organic Chemistry”, Jerry March, 4^{a} Edition, 1992, John Wiley & Sons Pub., pages 1216-1217 references cited therein.

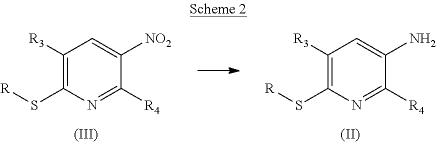

The preferred reaction conditions for these substrates include the use of tin chloride in concentrated hydrochloric acid, according to what is described in detail in international patent application WO 00/46184.

The compound having formula (III) can be obtained by reaction of the compound having formula (IV), wherein Y represents a bromine or chlorine atom, with a compound having formula RSH, in the presence of a base, such as sodium hydride or sodium methylate in an organic solvent such as tetrahydrofuran or N,N-dimethylformamide, according to reaction scheme 3.

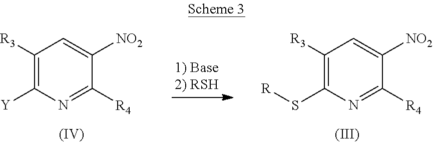

Alternatively, the compound having formula (III) can also be obtained by reaction of the compound having formula (IV), wherein Y represents a bromine or chlorine atom, with thiourea in the presence of an organic solvent such as ethanol or methanol, to obtain thiouronium salt; the latter, isolated or used as such in the reaction mixture, depending on the convenience of use, is reacted with a compound having formula RX, wherein X represents a chlorine, bromine or iodine atom, in the presence of a base, preferably sodium hydroxide, according to reaction scheme 4.

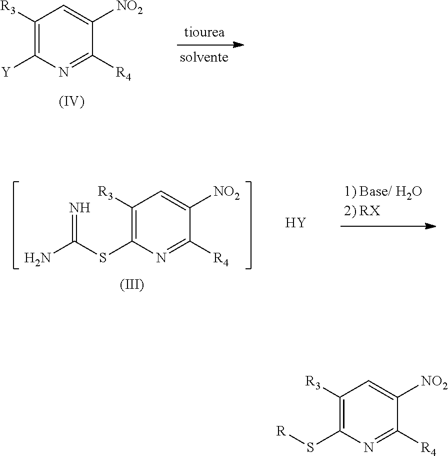

The compound having formula (IV) can be prepared according to what is described in detail in patent application US2010/029684.

As already specified, the compounds having general formula (I) have extremely high fungicidal activity which is exerted against numerous phytopathogenic fungi that attack important agricultural crops.

A further object of the present invention therefore relates to the use of the compounds having formula (I) for both the curative and preventive control of phytopathogenic fungi of agricultural crops.

Examples of phytopathogenic fungi that can be effectively treated and fought with the compounds having general formula (I) are those belonging to the classes of Basidiomycetes, Ascomycetes, Deuteromycetes or imperfect fungi, Oomycetes: Puccinia spp., Ustilago spp., Tilletia spp., Uromyces spp., Phakopsora spp., Rhizoctonia spp., Erysiphe spp., Sphaerotheca spp., Podosphaera spp., Uncinula spp., Helminthosporium spp., Rhynchosporium spp., Pyrenophora spp., Monilinia spp., Sclerotinia spp., Septoria spp. (Mycosphaerella spp.), Venturia spp., Botrytis spp., Alternaria spp., Fusarium spp., Cercospora spp., Cercosporella herpotrichoides, Colletotrichum spp., Pyricularia oryzae, Sclerotium spp., Phytophtora spp., Pythium spp., Plasmopara viticola, Peronospora spp., Pseudoperonospora cubensis, Bremia lactucae.

The main crops that can be protected with the compounds according to the present invention comprise cereals (wheat, barley, rye, oats, rice, corn, sorghum etc..), fruit-trees (apples, pears, plums, peaches, almonds, cherries, bananas, grapes, strawberries, raspberries, blackberries, etc.), citrus fruit (oranges, lemons, mandarins, grapefruit, etc.), legumes (beans, peas, lentils, soybeans, etc.), vegetables (spinach, lettuce, asparagus, cabbages, carrots, onions, tomatoes, potatoes, eggplants, peppers, etc.), cucurbits (pumpkins, courgettes, cucumbers, melons, watermelons, etc.), oil plants (sunflower, rapeseed, peanut, castor, coconut, etc.), tobacco, coffee, tea, cocoa, sugar beet, sugar cane, cotton. In particular, the compounds having formula (I) have proved to be extremely effective in the control of Plasmopara viticola on vines, Phytophtora infestans and Botrytis Cinerea on tomatoes, Puccinia Recondita, Erisiphae Graminis, Helminthosporium Teres, Parastagonospora nodorum, Zymoseptoria Tritici and Fusarium spp. on cereals, in the control of Phakopsora Pachyrhizi on soybeans, in the control of Uromyces Appendiculatus on beans, in the control of Venturia Inaequalis on apples, in the control of Sphaerotheca Fuliginea on cucumbers.

The compounds having general formula (I) have also proved to be effective in the control of phytopathogenic bacteria and viruses, such as, for example, Xanthomonas spp., Pseudomonas spp., Erwinia Amylovora, the tobacco mosaic virus. The present invention consequently also protects the use of compounds having formula (I) for the control of phytopathogenic bacteria and viruses, preferably those previously indicated.

The compounds having formula (I) are capable of exerting a fungicidal action of both a curative and preventive nature and show an extremely low or zero phytotoxicity with respect to the crops treated.

For practical uses in agriculture, it is often preferable to use fungicidal compositions containing the compounds according to the present invention suitably formulated.

A further object of the present invention relates to fungicidal compositions comprising one or more compounds having formula (I), a solvent and/or solid or liquid diluent, possibly a surfactant.

The above-mentioned fungicidal compositions can be in the form of dry powders, wettable powders, emulsifiable concentrates, emulsions, micro-emulsions, pastes, granules, granules dispersible in water, solutions, suspensions, etc.: the selection of the type of composition depends on the specific use.

The fungicidal compositions are prepared according to known methods, for example by diluting or dissolving the active substance with a solvent medium and/or a solid or liquid diluent, possibly in the presence of surfactants.

Silica, kaolin, bentonite, talc, diatomaceous earth, dolomite, calcium carbonate, magnesia, gypsum, clays, synthetic silicates, attapulgite, seppiolite, can be used as solid diluents, or carriers.

Solvents or liquid diluents that can be used for example, in addition to water, are aromatic organic solvents (xylols or blends of alkyl benzenes, chlorobenzene, etc.), paraffins (petroleum fractions), alcohols (methanol, propanol, butanol, octanol, glycerin, etc.), esters (ethyl acetate, isobutyl acetate, 2-ethylhexyl acetate, alkyl carbonates, alkyl esters of adipic acid, alkyl esters of glutaric acid, alkyl esters of succinic acid, alkyl esters of lactic acid, etc.), vegetable oils (rapeseed oil, sunflower oil, soybean oil, castor oil, corn oil, peanut oil, and their alkyl esters), ketones (cyclohexanone, acetone, acetophenone, isophorone, ethylamylketone, etc.), amides (N, N-dimethylformamide, N-methylpyrrolidone, etc.), sulfoxides and sulfones (dimethyl sulfoxide, dimethyl-sulfone, etc.), and mixtures thereof.

Surfactants that can be used are sodium, calcium, potassium, triethylamine or triethanolamine salts of alkylnaphthalenesulfonates, poly-naphthalenesulfonates, alkylsulfonates, arylsulfonates, alkylarylsulfonates, polycarboxylates, sulfosuccinates, alkyl-sulfosuccinates, lignin sulfonates, alkyl sulfates; and furthermore polyethoxylated fatty alcohols, polyethoxylated alkylphenols, polyethoxylated or polypropoxy-polyethoxylated arylphenols ror esters of polyethoxylated sorbitol, polyproproxy-polyethoxylates (block polymers) can also be used.

The fungicidal compositions can also contain special additives for particular purposes, for example antifreeze agents such as propylene glycol, or tackifying agents such as arabic gum, polyvinyl alcohol, polyvinylpyrrolidone, etc.

Fungicidal compositions for the compounds having general formula (I) that are particularly preferred for their high stability to light and heat over time, as described in Example 17 of the present patent application, and which can therefore be effectively used in agronomic practice, are formulated as an emulsifiable concentrate based on propylene carbonate, N,N-dimethyloctanamide, N,N-dimethyldecanamide, acetophenone, 2-ethylhexyl acetate, alkyl esters of adipic acid, alkyl esters of glutaric acid, alkyl esters of succinic acid, dimethyl sulfoxide or based on morpholine solvents, preferably N-fortnylinorpholine, alone or mixed with each other, in a quantity ranging from 2% to 60% by weight with respect to the total weight of the fungicidal composition.

Preferred surfactants are selected from sodium, calcium or potassium alkaryl sulfonates, preferably calcium dodecylbenzenesulfonate, or polyethoxylated or polypropoxy-polyethoxylated arylphenols, preferably ethoxylated-propoxylated polyarylphenols.

If desired, other active ingredients can be added to the fungicidal compositions containing the compounds having general formula (I), compatible with the same, selected from fungicides different from those having general formula (I), plant growth regulators, antibiotics, herbicides, insecticides, fertilizers, biostimulants and/or mixtures thereof, preferably fungicides.

Examples of fungicides different from those having general formula (I) that can be included in the fungicidal compositions object of the present invention are: fluindapyr, acibenzolar, ametoctradin, amisulbrom, ampropylfos, anilazine, azaconazole, azoxystrobin, benalaxyl, benalaxyl-M, benomyl, benthiavalicarb, bitertanol, bixafen, blasticidin-S, boscalid, bromuconazole, bupirimate, buthiobate, captafol, captan, carbendazim, carboxin, carpropamid, chinomethionat, chloroneb, chlorothalonil, chlozolinate, cyazofamid, cyflufenamid, cymoxanil, cyproconazole, cyprodinil, debacarb, dichlofluanid, dichlone, diclobutrazol, diclomezine, dicloran, diclocymet, diethofencarb, difenoconazole, diflumetorim, dimethirimol, dimethomorph, dimoxystrobin, diniconazole, dinocap, dipyrithione, ditalimfos, dithianon, dodemorph, dodine, edifenphos, epoxiconazole, etaconazole, ethaboxam, ethirimol, ethoxyquin, etridiazole, famoxadone, fenamidone, fenaminosulf, fenapanil, fenarimol, fenbuconazole, fenfuram, fenhexamid, fenoxanil, fenpiclonil, fenpropidin, fenpropimorph, fenpyrazamine, fentin, ferbam, ferimzone, fluazinam, fludioxonil, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpet, fosetyl-aluminium, fuberidazole, furalaxyl, furametpyr, furconazole, furconazole-cis, guazatine, hexaconazole, hymexazol, hydroxyquinoline sulfate, imazalil, imibenconazole, iminoctadine, ipconazole, iprobenfos, iprodione, isoprothiolane, iprovalicarb, isopyrazam, isotianil, kasugamycin, kresoxim-methyl, mancopper, mancozeb, mandipropamid, maneb, mebenil, mepanipyrim, mepronil, meptyldinocap, metalaxyl, metalaxyl-M, metconazole, methfuroxam, metiram, metominostrobin, metrafenone, metsulfovax, myclobutanil, natamycin, nicobifen, nitrothal-isopropyl, nuarimol, ofurace, orysastrobin, oxadixyl, oxpoconazole, oxycarboxin, pefurazoate, penconazole, pencycuron, penflufen, pentachlorofenol and its salts, penthiopyrad, phthalide, picoxystrobin, piperalin, Bordeaux mixture, polyoxins, probenazole, prochloraz, procymidone, propamocarb, propiconazole, propineb, proquinazid, prothiocarb, prothioconazole, pyracarbolid, pyraclostrobin, pyrametostrobin, pyraoxystrobin, pyrazophos, pyribencarb, pyrifenox, pyrimethanil, pyriofenone, pyroquilon, pyroxyfur, quinacetol, quinazamid, quinconazole, quinoxyfen, quintozene, rabenzazole, copper hydroxide, copper oxychloride, copper (I) oxide, copper sulfate, sedaxane, silthiofam, simeconazole, spiroxamine, streptomycin, tebuconazole, tebufloquin, tetra-conazole, thiabendazole, thiadifluor, thicyofen, thifluzamide, thiophanate, thiophanate-methyl, thiram, tiadinil, tioxymid, tolclofos-methyl, tolylfluanid, triadimefon, triadimenol, triarimol, triazbutil, triazoxide, tricyclazole, tridemorf, trifloxystrobin, triflumizole, triforine, triticonazole, uniconazole, uniconazole-P, validamycin, valifenalate, vinclozolin, zineb, ziram, sulfur, zoxamide.

A further object of the present invention therefore relates to fungicidal compositions comprising at least one compound having general formula (I) and at least one other known fungicide.

Fungicidal compositions containing at least one pyridyl-formamidine having formula (I) and one or more known fungicides, which are especially preferred for the particularly broad spectrum of action and a strong synergistic effect, are those wherein one or more compounds having general formula (I) are combined with one or more known fungicides belonging to the following classes:

a) azoles selected from azaconazole, bitertanol, bromuconazole, cyproconazole, difenoconazole, epoxyconazole, fenbuconazole, fluquinconazole, flusilazole, flutriafol, hexaconazole, imazalil, ipconazole, metconazole, myclobutanil, penconazole, propiconazole, prochloraz, prothioconazole, simeconazole tebuconazole, tetraconazole, triadimefon, triadimenol, triflumizole, triticonazole;

b) amines, ergosterol biosynthesis inhibitors selected from aldimorph, dodemorph, fenpropimorph, fenpropidin, spiroxamine, tridemorph;

c) succinate-dehydrogenase inhibitors (SDHI) selected from benzovindiflupyr, bixafen, boscalid, carboxin, fluindapyr, fluopyram, flutolanil, fluxapyroxad, furametpyr, isopyrazam, oxycarboxin, penflufen, penthiopyrad, sedaxane, thifluzamide;

d) strobilurins selected from azoxystrobin, dimoxystrobin, fluoxastrobin, kresoxim-methyl, metominostrobin, orysastrobin, picoxystrobin, pyraclostrobin, pyrametostrobin, pyraoxostrobin, trifloxystrobin;

e) specific antioidic compounds selected from cyflufenamid, flutianil, metrafenone, proquinazid, pyriofenone, quinoxyfen;

f) aniline-pyramidines selected from pyrimethanil, mepanipyrim, cyprodinil;

g) benzimidazoles and analogues thereof selected from carbendazim, benomyl, thiabendazole, thiophanate-methyl;

h) dicarboxyimides selected from iprodione, procymidone;

i) phtalimides selected from captafol, captan, folpet;

l) systemic acquired resistance (SAR) inducers selected from acibenzolar, probenazole, isotianil, tiadinil;

m) phenylpyrroles selected from fenpiclonil, fludioxonil;

n) acylalanines selected from benalaxyl, benalaxyl-M, furalaxyl, metalaxyl, metalaxyl-M;

o) other specific antiperonosporic compounds selected from ametoctradin, amisulbrom, benthiavalicarb, cyazofamid, cymoxanil, dimethomorph, ethaboxam, famoxadone, fenamidone, flumetover, flumorph, fluopicolide, iprovalicarb, mandipropamid, oxathiapiproline, vali-fenalate;

p) dithiocarbamates selected from maneb, mancozeb, propineb, zineb;

q) phosphorous acid and its inorganic or organic salts, fosetyl-aluminium;

r) rameic compounds selected from Bordeaux mixture, carpropamid, copper hydroxide, copper oxychloride, copper sulfate, copper salycilate;

s) other fungicides selected from chlorothalonil, fenhexamid, fenpyrazamine, fluazinam, sylthiofam, tebufloquin, zoxamide, dodine, guazatine, iminoctadine.

The fungicidal compounds are indicated in the present description with their ISO international name; the chemical structures and their CAS and IUPAC names are indicated in Alan Wood's Web site (www.alanwood.net), Compendium of Pesticide Common Names; the physico-chemical data and biological characteristics of most of these compounds are specified in the “Pesticide Manual”, C.D.S. Tomlin, 15^{th} Edition, 2009, British Crop Production Council.

Preferred compositions, containing at least one compound having formula (I) (component A) and at least another known fungicide, are those consisting of:
C1: compound 3+tetraconazole;
C2: compound 3+tebuconazole;
C3: compound 3+epoxyconazole;
C4: compound 3+prothioconazole;
C5: compound 3+prochloraz;
C6: compound 3+fenpropimorph;
C7: compound 3+spiroxamine;
C8: compound 3+bixafen;
C9: compound 3+boscalid;
C10: compound 3+carboxin;
C11: compound 3+fluopyram;
C12: compound 3+fluxapyroxad;
C13: compound 3+isopyrazam;
C14: compound 3+penthiopyrad;
C15: compound 3+sedaxane;
C16: compound 3+azoxystrobin;
C17: compound 3+dimoxystrobin;
C18: compound 3+fluoxastrobin;
C19: compound 3+kresoxim-methyl;
C20: compound 3+picoxystrobin;
C21: compound 3+pyraclostrobin;
C22: compound 3+trifloxystrobin;
C23: compound 3+metrafenone;
C24: compound 3+proquinazid;
C25: compound 3+mepanipyrim;
C26: compound 3+cyprodinil;
C27: compound 3+iprodione;
C28: compound 3+procymidone;
C29: compound 3+carbendazim;
C30: compound 3+thiophanate-methyl;
C31: compound 3+fluindapyr;
C32: compound 3+benalaxyl-M;
C33: compound 3+benzovindiflupyr;
C34: compound 1+tetraconazole;
C35: compound 1+fluindapyr;
C36: compound 1+azoxystrobin;
C37: compound 1+pyraclostrobin;
C38: compound 2+tetraconazole;
C39: compound 2+tebuconazole;
C40: compound 2+epoxyconazole;
C41: compound 2+prothioconazole;
C42: compound 2+prochloraz;
C43: compound 2+fenpropimorph;
C44: compound 2+spiroxamine;
C45: compound 2+bixafen;
C46: compound 2+boscalid;
C47: compound 2+carboxin;
C48: compound 2+fluopyram;
C49: compound 2+fluxapyroxad;
C50: compound 2+isopyrazam;
C51: compound 2+penthiopyrad;
C52: compound 2+sedaxane;
C53: compound 2+azoxystrobin;
C54: compound 2+dimoxystrobin;
C55: compound 2+fluoxastrobin;
C56: compound 2+kresoxim-methyl;
C57: compound 2+picoxystrobin;
C58: compound 2+pyraclostrobin;
C59: compound 2+trifloxystrobin;
C60: compound 2+metrafenone;
C61: compound 2+proquinazid;
C62: compound 2+mepanipyrim;
C63: compound 2+cyprodinil;
C64: compound 2+iprodione;
C65: compound 2+procymidone;
C66: compound 2+carbendazim;
C67: compound 2+thiophanate-methyl;
C68: compound 2+fluindapyr;
C69: compound 2+benalaxyl-M;
C70: compound 2+benzovindiflupyr;
C71: compound 2+tetraconazole+azoxystrobin,
C72: compound 2+pyraclostrobin+tetraconazole;
C73: compound 2+epoxyconazole+azoxystrobin;
C74: compound 2+pyraclostrobin+epoxyconazole;
C75: compound 3+azoxystrobin+fluindapyr;
C76: compound 3+pyraclostrobin+fluindapyr;
C77: compound 3+fluindapyr+tetraconazole;
C78: compound 3+tetraconazole+azoxystrobin;
C79: compound 3+pyraclostrobin+tetraconazole;
C80: compound 3+azoxystrobin+fluindapyr;
C81: compound 3+fluindapyr+tetraconazole.

Component A, i.e. the compounds having general formula (I), of the above-mentioned compositions C1-C81 are described and exemplified in Table 1 and are specifically the following compounds having general formula (I) wherein the substituents have the meanings indicated hereunder:

| Compound | | | | | |
|---|---|---|---|---|---|
| Nr. | R | R₁ | R₂ | R₃ | R₄ |
| 1. | 3-CF₃-benzyl | CH₃ | Et | Br | CH₃ |
| 2. | 3-methyl-butyl | CH₃ | Et | CH₃ | CH₃ |
| 3. | 3-CF₃-benzyl | CH₃ | Et | CH₃ | CH₃ |

The synergistic effect of the compositions containing a compound having general formula (I) (component A) and a known fungicide (component B), can be evaluated by applying the Colby formula (“Weeds, 1967, 15, pages 20-22):

Eₜ=E_{A}+E_{B}−(E_{A}×E_{B}):100

wherein Eₜ is the expected percentage of effectiveness for the composition containing compounds A and B at the doses d_{A}+d_{B}, E_{A} is the percentage of effectiveness observed for component A at the dose d_{A}, E_{B} is the percentage of effectiveness observed for component B at the dose d_{B}.

When the effectiveness observed for the composition A+B (E_{A+B}) is higher than the expected effectiveness according to the Colby formula (E_{A+B}/Eₜ>1), there is a synergistic effect.

In the case of ternary combinations, the Colby formula has the form:

Eₜ=E_{A}+E_{B1}+E_{B2}−(E_{A}×E_{B1}+E_{A}×E_{B2}+E_{B1}×E_{B2})/100

wherein Eₜ is the expected percentage of effectiveness for the composition containing compounds A, B1 and B2 at the doses d_{A}+d_{B1}+d_{B2}, E_{A} is the percentage of effectiveness observed for component A at the dose d_{A}, E_{B1} is the percentage of effectiveness observed for component B1 at the dose d_{B1}, E_{B2} is the percentage of effectiveness observed for component B2 at the dose d_{B2}.

When the percentage of effectiveness observed for the composition A+B1+B2(E_{A+B1+B2}) is higher than the expected effectiveness according to the Colby formula (E_{A+B1+B2}/Eₜ>1), there is a synergistic effect.

The main crops that can be protected with the compositions comprising at least one compound having formula (I), alone or combined with at least one other known active ingredient, comprise cereals (wheat, barley, rye, oats, rice, corn, sorghum, etc.), fruit (apples, pears, plums, peaches, almonds, cherries, bananas, grapes, strawberries, raspberries, blackberries, etc.), citrus fruits (oranges, lemons, mandarins, grapefruit, etc.), legumes (beans, peas, lentils, soybeans, etc.), vegetables (spinach, lettuce, asparagus, cabbage, carrots, onions, tomatoes, potatoes, eggplants, peppers, etc.), cucurbits (pumpkins, zucchini, cucumbers, melons, watermelons, etc.), oleaginous plants (sunflowers, rapeseed, peanuts, castor, coconut. etc.); tobacco, coffee, tea, cocoa, sugar beet, sugar cane, cotton, nuts.

In particular, the compositions of the present invention have proved to be considerably effective in the control of Plasmopara viticola on vines, Phytophtora infestans and Botrytis Cinerea on tomatoes, Puccinia Recondita, Erisiphae Graminis, Helminthosporium Teres, Septoria spp. and Fusarium spp. on cereals, in the control of Phakopsora Pachyrhizi on soybeans, in the control of Uromyces Appendiculatus on beans, in the control of Venturia Inaequalis on apples, in the control of Sphaerotheca Fuliginea on cucumbers.

Furthermore, the compositions of the present invention are also effective in the control of phytopathogenic bacteria and viruses, preferably Xanthomonas spp., Pseudomonas spp., Erwinia Amylovora, the tobacco mosaic virus.

The compositions, object of the present invention, are capable of exerting a fungicidal action that can be of a curative, preventive or eradicative nature, and, in general, exhibit a very low or zero phytotoxicity on the crops treated.

A further object of the present invention therefore relates to the use of the compositions comprising at least one compound having general formula (I) for the control of phytopathogenic fungi in agricultural crops.

If the compositions comprise a compound having general formula (I) and at least one known active ingredient, the weight ratios in the above compositions vary according to the compounds selected and can normally range from 1:100 to 100:1, preferably from 1:10 to 10:1.

The total concentration of the active components in the above compositions can vary within a wide range; they generally range from 1% to 99% by weight with respect to the total weight of the composition, preferably from 5 to 90% by weight with respect to the total weight of the composition.

The application of these compositions can take place on every part of the plant, for example on the leaves, stems, branches and roots, or on the seeds themselves before sowing, or on the soil where the plant grows.

A further object of the present invention therefore relates to a method for controlling phytopathogenic fungi in agricultural crops, which consists in applying effective and non-phytotoxic doses of compounds having formula (I), used as such or formulated in fungicidal compositions as described above, i.e. compositions comprising at least one compound having general formula (I) and, optionally, one or more known active ingredients compatible with the same.

The concentration of the formamidine compounds having general formula (I) in the above-mentioned compositions can vary within a wide range; in general, it ranges from 1% to 90% by weight with respect to the total weight of the composition, preferably from 5 to 50% by weight with respect to the total weight of the composition.

The application of these compositions can take place on every part of the plant, for example on the leaves, stems, branches and roots, or on the seeds themselves before sowing, or on the soil where the plant grows.

The quantity of compound to be applied for obtaining the desired effect can vary according to various factors such as, for example, the compound used, the crop to be preserved, the type of pathogen, the degree of infection, the climatic conditions, the method of application, the formulation adopted.

Doses of compound ranging from 10 g to 5 kg per hectare of agricultural crop generally provide a sufficient control.

The following examples are provided for a better understanding of the invention, which are to be considered illustrative and non-limitative of the same.

### EXAMPLE 1

Preparation of 3,6-dimethyl-2-(3-methylbutyl)thio-4-nitropyridine.

[(Nitroderivative having general formula (III)}

A solution of 6.6 g of 3-methylbutyl-1-thiol (35.4 mmoles) and 3.6 g of 2-chloro-3,6-dimethyl-5-nitropyridine (35.4 mmoles) in (88.5 ml) of tetrahydrofuran was cooled with an ice bath to 0° C., and 1.9 g of sodium hydride (82.6 mmoles) at 60% were added in small portions. The reaction was allowed to reach room temperature and was then left under stirring at this temperature for 24 hours.

After control with GC-MS and LC-MS, the reaction mixture was diluted with water and the phases were separated. The organic phase was re-extracted with ethyl acetate, washed with water and subsequently with a saturated solution of sodium chloride, anhydrified on sodium sulfate, filtered and evaporated, to give 8.5 g of product.

The product thus obtained was purified by silica gel chromatography, eluting with hexane/ethyl acetate 9:1. 6.5 g of the desired product were obtained.

GC-MS: M⁺=254.

### Example 2

Preparation of 2,5-dimethyl-6-[(3-methylbutypthio]pyridyl-3-amine.

[Pyridylamine having general formula (II)]

2.1 ml of glacial acetic acid (37.61 mmoles) were added to a solution of 6.5 g (25.5 mmoles) of 3,6 dimethyl-2-(3-methylbutyl)thio-4-nitropyridine in water (10 ml) and ethanol (100 ml); 9.6 g of Fe in powder form (172.7 mmoles) were added with caution to the reaction mixture, kept under stirring at 60° C. The temperature was brought to 90° C. and the reaction was kept under stirring for 1.5 hours. After control with GC-MS and LC-MS, the mixture was cooled to room temperature, filtered on celite and concentrated at reduced pressure. The product thus obtained was washed with a saturated solution of sodium bicarbonate and extracted with ethyl acetate. The organic phase was washed with water anhydrified on sodium sulfate, filtered and evaporated to give 4.8 g of the desired product.

GC-MS: M⁺=224.

### Example 3

Preparation of N-ethyl-N-methyl-N′-(2,5-dimethyl-6-[(3-methylbutyl) thio]pyridyl-3-formamidine.

[Compound 2].

Catalytic p-toluenesulfonic acid was added to a mixture of 4.8 g (21.4 mmoles) of 2,5-dimethyl-6-[(3-methylbutyl)thio]pyridyl-3-amine and 17.7 ml of triethylorthoformiate (107.1 mmoles). The reaction mixture was brought to a temperature of 100° C. and kept under stirring for 1 hour. The reaction trend was monitored by means of GC-MS. When completed, the reaction mixture was concentrated at reduced pressure and the raw product obtained was dissolved in methylene chloride (16.4 ml). 2.7 ml of N-ethyl-N-methylamine (32.1 mmoles) were subsequently added dropwise. The mixture was left under stirring for 24 hours and, after control with GC-MS, was subsequently diluted with water, and the phases were separated. The aqueous phase was re-extracted with methylene chloride, the combined organic phases were washed with water, with a saturated solution of sodium chloride, anhydrified on sodium sulfate, filtered and evaporated. 5.6 g of the desired product were obtained.

GC-MS: M+=295; LC-MS 98%.

¹H-NMR (CDCl₃) δ=0.86 (d, 6 H); 1.19 (t, 3H); 1.45-1.48 (m ,2H); 2.06 (s, 3H); 2.27 (s, 3H); 2.91 (s, 3H); 3.15 (t, 2H); 3.48 (q ,2H); 7.06 (s, 1H); 8.51 (s, 1H).

### Example 4

Preparation of 3,6-dimethyl-4-nitro-{[3(trifluorome-thyl)benzyl]thio}pyridine.

[Nitroderivative having general formula (III)].

A solution of 7.8 g of 3-trifluoromethyl-benzyl-1-thiol (40.9 mmoles) and 7.2 g of 2-chloro-3,6-dimethyl-5-nitropyridine (38.9 mmoles) in tetrahydrofuran (130 ml) was cooled with an ice bath to 0° C. 2.1 g of sodium hydride (90.6 mmoles) at 60% were then added in small portions. The reaction was allowed to reach room temperature and left under stirring at this temperature for 24 hours.

After controlling the reaction trend with GC-MS and LC-MS, the reaction mixture was diluted with water and the phases were separated. The organic phase was re-extracted with ethyl acetate, washed with water and subsequently with a saturated solution of sodium chloride, anhydrified on sodium sulfate, filtered and evaporated to give 12.9 g of product.

GC-MS: M+=342

### Example 5

Preparation of 2,5-dimethyl-6-{[3(trifluoromethyl) benzyl]thio}pyridyl-3-amine [Pyridylamine having general formula (II)].

3.2 ml of glacial acetic acid (55.4 mmoles) were added to a solution of 12.9 g of 3,6-dimethyl-4-nitro-2-{[3 (trifluoromethyl)benzyl]thio}pyridine (37.7 mmoles) in water (15 ml) and ethanol (150 ml). 14.2 g of Fe in powder form (254.4 mmoles) were added with caution to the reaction mixture, kept under stirring at 60° C.; the temperature was then increased to 90° C. and the reaction was kept under stirring for 1.5 hours.

After controlling the reaction trend with GC-MS and LC-MS, the mixture was cooled to room temperature and filtered on celite. Most of the ethanol was evaporated at reduced pressure and the product obtained was washed with a saturated solution of sodium bicarbonate and then extracted with ethyl acetate. The organic phase was washed with water, anhydrified on sodium sulfate, filtered and evaporated to give 11.5 g of product.

GC-MS: M^{±}=312.

### Example 6

Preparation of N-ethyl-N-methyl-N′-2,5-dimethyl-6-{[3(trifluoromethyl)benzyl]thio}pyridyl-3-formamidine [Compound 3].

Catalytic p-toluenesulfonic acid was added to a mixture of 11.5 g (36.8 mmoles) of 2,5-dimethyl-6-{[3 (tri-fluoromethyl)benzyl]thio}pyridyl-3-amine and 30 ml of triethylorthoformiate (184.2 mmoles). The temperature was brought to 100° C. and the reaction kept under stirring for 1 hour, controlling the reaction trend with GC-MS. The reaction mixture was then concentrated at reduced pressure, the raw product obtained was dissolved in methylene chloride (28.3 ml) and 4.6 ml of N-ethyl-N-methylamine (55.3 mmoles) were subsequently added dropwise to the reaction mixture. The mixture was kept under stirring at room temperature for 24 hours and the reaction trend was then controlled with GC-MS. The solvent was subsequently evaporated at reduced pressure and the product thus obtained was purified by silica gel chromatography, eluting with a mixture of hexane/ethyl acetate 98:2 containing 1% of triethylamine. 10.4 g of the desired product were obtained.

GC-MS: M+=381 LC-MS 97.3%.

¹H-NMR (CDCl₃) δ=1.19 (t, 3H); 2.06 (s, 3H); 2.27 (s, 3H); 2.91 (s, 3H); 3.5 (q, 2H); 4.3 (s, 2H); 7.06 (s, 1H); 6.95-7.18 (m, 4H); 8.51 (s, 1H).

### Example 7

Preparation of N-ethyl-N-methyl-N′-5bromo-2-methyl-6-{[3-(trifluoromethyl)benzyl]thio}pyridyl-3-formamidine. [Compound 1].

Analogously to what is described in Examples 4, 5 and 6, 4.5 g of the desired product were obtained, starting from 7.5 g of 3-bromo-2-chloro-6-methyl-5-nitro-pyridine (29.82 mmoles) and 6.0 g of 3-(trifluoromethyl)-benzyl-1-thiol (29.82 mmoles).

GC-MS: M+=446 LC-MS 98.3%.

¹H-NMR (CDCl₃) δ=1.19 (t, 3H); 2.06 (s, 3H); 2.91 (s, 3H); 3.5 (q, 2H); 4.0 (s, 2H); 7.06 (s, 1H); 6.95-7.18 (m, 4H); 8.51 (s, 1H).

### Example 8

Preparation of 3,6-dimethyl-2-[(3-methylbenzyl)thio]-nitropyridine

[Nitroderivative having general formula (III)].

0.4 g of thiourea (5.3 mmoles) were added in a nitrogen stream to 1 g of 2-chloro-3,6-dimethyl-5-nitropyridine (5.3 mmoles), dissolved in ethanol (7.6 ml).

The reaction temperature was brought to reflux and maintained for three hours, the reaction was then controlled in LC-MS. The reaction mixture was cooled to room temperature and a solution of 0.51 g of sodium hydroxide (12.8 mmoles) in 10 ml of water was subsequently added. After 30 minutes, 0.7 ml of 3-(methyl)-benzylbromide (5.3 mmoles) dissolved in 1 ml of ethanol were slowly added dropwise and the whole mixture was refluxed for two hours.

After control with LC-MS, the reaction was diluted with water and extracted with ethyl acetate; the organic phase was anhydrified with sodium sulfate and evaporated at reduced pressure, obtaining 1.8 g of product.

GC-MS: M+=288

### Example 9

Preparation of 2,5-dimethyl-6-[(3-(methyl)benzyl)thio]pyridyl-3-amine.

[Pyridylamine having general formula (II)].

0.4 ml of glacial acetic acid (7.5 mmoles) were added to a solution of 1.8 g of 3,6-dimethyl-2-[(3-(methyl)benzyl)thio]-4-nitropyidine (6.2 mmoles) in water (2.6 ml) and ethanol (26 ml); 1 g of Fe in powder form (18.75 mmoles) were added with caution to the reaction mixture kept under stirring at 60° C. The temperature was brought to 90° C. and the reaction mixture was left under stirring at this temperature for 1.5 hours. After controlling the reaction in GC-MS and LC-MS, the mixture was cooled to room temperature and filtered on celite. The mixture was concentrated at reduced pressure and the product thus obtained was washed with a saturated solution of sodium bicarbonate and extracted with ethyl acetate. The organic phase, washed with water, was anhydrified on sodium sulfate, filtered and evaporated at reduced pressure, obtaining 1.6 g of product.

GC-MS: M+=258.

### Example 10

Preparation of N-ethyl-N-methyl-N′-{2,5-dimethyl-6-[(3-methylbenzyl)thio]pyridyl-3-formamidine.

[Compound 4]

Catalytic p-toluenesulfonic acid was added to a mixture of 1.6 g (6.2 mmoles) of 2,5-dimethyl-6-[(3-methylbenzyl)thio]pyridyl-3-amine and 5.1 ml of triethylorthoformiate (31.0 mmoles). The temperature was brought to 100° C. and the reaction kept under stirring at this temperature for 1 hour. The reaction trend was controlled with GC-MS. When completed, the reaction mixture was concentrated at reduced pressure and the raw material obtained was dissolved in methylene chloride (4.3 ml). 0.7 ml of N-ethyl-N-methylamine (9.3 mmoles) were added dropwise and the whole mixture was then left under stirring at room temperature for 24 hours. After control in GC-MS, the solvent was evaporated at reduced pressure and the raw material thus obtained was purified by silica gel chromatography, eluting with a mixture of hexane/ethyl acetate 9:1, with the addition of 1% of triethylamine. 0.7 g of the desired product were obtained.

GC-MS: M+=327 LC-MS 95%.

¹H-NMR (CDCl₃) δ=1.19 (t, 3H); 2.06 (s, 3H); 2.27 (s, 3H); 2.31 (s,3H);); 2.91 (s, 3H); 3.5 (q, 2H); 4.27 (s, 2H); 7.06 (s, 1H); 6.95-7.18 (m, 4H); 8.51 (s, 1H).

### Example 11

Preparation of Compounds 5-83

Compounds 5-83 having formula (I) indicated in Table 2 were obtained operating analogously to what is described in the previous examples.

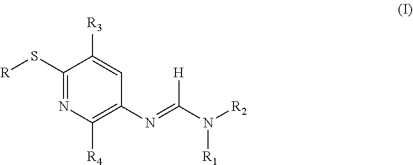

TABLE 2

| Compound | | | | | |
|---|---|---|---|---|---|
| Nr. | R | R1 | R2 | R3 | R4 |
| 5. | 3-methyl-1-butyl | CH₃ | Et | Br | CH₃ |
| 6. | 3-CF₃-phenyl | CH₃ | Et | Br | CH₃ |
| 7. | 3-CF₃-phenyl | CH₃ | Et | H | CH₃ |
| 8. | 4-Cl-3-CF₃-phenyl | CH₃ | Et | Br | CH₃ |
| 9. | 3-CF₃-phenyl | CH₃ | Et | CH₃ | CH₃ |
| 10. | 3-propoxy-2-propyl | CH₃ | Et | Br | CH₃ |
| 11. | 3-methyl-1-butyl | CH₃ | Et | H | CH₃ |
| 12. | 3-propoxy-2-propyl | CH₃ | Et | CH₃ | CH₃ |
| 13. | 5-CH₃-1,3,4-thiadiazol-2-yl | CH₃ | Et | Br | CH₃ |
| 14. | 3-CF₃-phenyl | CH₃ | Et | Br | OMe |
| 15. | 3,4,4-trifluorobut-3-en-1-yl | CH₃ | Et | CH₃ | CH₃ |
| 16. | benzyl | CH₃ | Et | CH₃ | CH₃ |
| 17. | n-hexyl | CH₃ | Et | CH₃ | CH₃ |
| 18. | 4-Cl-3-CF₃-benzyl | CH₃ | Et | CH₃ | CH₃ |
| 19. | 2-Fluoro-benzyl | CH₃ | Et | CH₃ | CH₃ |
| 20. | 3-methyl-thiazol-2-yl | CH₃ | Et | CH₃ | CH₃ |
| 21. | 2-ethyl-1-hexyl | CH₃ | Et | CH₃ | CH₃ |
| 22. | 3-CF₂H-benzyl | CH₃ | Et | CH₃ | CH₃ |
| 23. | n-decyl | CH₃ | Et | CH₃ | CH₃ |
| 24. | 1-adamantyl | CH₃ | Et | CH₃ | CH₃ |
| 25. | cyclododecyl | CH₃ | Et | Br | CH₃ |
| 26. | 5-Br-2-CH₃-thiazol-4-yl | CH₃ | Et | CH₃ | CH₃ |
| 27. | (3-tertbutyl-5-isoxazolyl)methyl | CH₃ | Et | CH₃ | CH₃ |
| 28. | 2-isopropoxycarbonyl-1-ethyl | CH₃ | Et | CH₃ | CH₃ |
| 29. | 5,5-dimethyl-3-methoxy- | CH₃ | Et | Br | CH₃ |
| | imino-1-pentyl | | | | |
| 30. | 2-(cyclopropyl- | CH₃ | Et | CH₃ | CH₃ |
| | aminocarbonyl)-1-ethyl | | | | |
| 31. | 4,4-dimethyl-3-oxa-1-pentyl | CH₃ | Et | Br | CH₃ |
| 32. | (2,2-dichloro-1-methyl- | CH₃ | Et | CH₃ | CH₃ |
| | cyclopropyl)-methyl | | | | |
| 33. | 3-(ethylthio)-1-butyl | CH₃ | Et | CH₃ | CH₃ |
| 34. | 3-[3-(2-oxa-propyl)- | CH₃ | Et | CH₃ | CH₃ |
| | cyclohexyl]-1-propyl | | | | |
| 35. | 5,5-dimethyl-3-isoxazolyl | CH₃ | Et | Br | CH₃ |
| 36. | (2-naphthyl)methyl | CH₃ | Et | CH₃ | CH₃ |
| 37. | 2-phenyl-1-ethyl | CH₃ | Et | CH₃ | CH₃ |
| 38. | cyclopropanoyl | CH₃ | Et | CH₃ | CH₃ |
| 39. | 2,6-difluorobenzyl | CH₃ | Et | CH₃ | CH₃ |
| 40. | cyclohexylmethyl | CH₃ | Et | CH₃ | CH₃ |
| 41 | 3-CF₃-phenyl | CH₃ | Et | Br | Cl |
| 42 | 3-methyl-1-butyl | CH₃ | Et | Br | Br |
| 43 | 2-fluoro-3-CF₃-benzyl | CH₃ | Et | CH₃ | CH₃ |
| 44 | 3-CF₃-phenyl | CH₃ | Et | Br | Br |
| 45 | 4-t-butyl-benzyl | CH₃ | Et | CH₃ | CH₃ |
| 46 | 1-adamantyl-methyl | CH₃ | Et | CH₃ | CH₃ |
| 47 | 2-fluoro-4,6 diCF₃-benzyl | CH₃ | Et | CH₃ | CH₃ |
| 48 | 4-fluoro-benzyl | CH₃ | Et | CH₃ | CH₃ |
| 49 | 4-F-3-CF₃-benzyl | CH₃ | Et | CH₃ | CH₃ |
| 50 | 1-phenyl-ethyl | CH₃ | Et | CH₃ | CH₃ |
| 51 | 2-iodo-benzyl | CH₃ | Et | CH₃ | CH₃ |
| 52 | cyclohexyl | CH₃ | Et | CH₃ | CH₃ |
| 53 | 2-CH₃-benzyl | CH₃ | Et | CH₃ | CH₃ |
| 54 | 2-CF₃-benzyl | CH₃ | Et | CH₃ | CH₃ |
| 55 | 2-fluoro-6-CF₃-benzyl | CH₃ | Et | CH₃ | CH₃ |
| 56 | 2-chloro-benzyl | CH₃ | Et | CH₃ | CH₃ |
| 57 | 2-bromo-benzyl | CH₃ | Et | CH₃ | CH₃ |
| 58 | 2-(cyclohexyl)-ethyl | CH₃ | Et | CH₃ | CH₃ |
| 59 | cyclooctyl | CH₃ | Et | CH₃ | CH₃ |
| 60 | 2-Br-6-OCF₂H-benzyl | CH₃ | Et | CH₃ | CH₃ |
| 61 | 2-Br-6-CF₃-benzyl | CH₃ | Et | CH₃ | CH₃ |
| 62 | 2-OCF₂CF₂H-6-CF₃-benzyl | CH₃ | Et | CH₃ | CH₃ |
| 63 | cyclopentylmethyl | CH₃ | Et | CH₃ | CH₃ |
| 64 | cyclopentyl | CH₃ | Et | CH₃ | CH₃ |
| 65 | 1-methyl-3-CF₃-5-OCH₂CF₃- | CH₃ | Et | CH₃ | CH₃ |
| | 4-pyrazolylmethyl | | | | |
| 66 | 4-pyridylmethyl | CH₃ | Et | CH₃ | CH₃ |
| 67 | 2-pyridylmethyl | CH₃ | Et | CH₃ | CH₃ |
| 68 | 5,5 dimethyl-2 isoxazolyn-3 yl | CH₃ | Et | CH₃ | CH₃ |
| 69 | 5-CH₃-1,3,4-thiadiazol-2-yl | CH₃ | Et | CH₃ | CH₃ |
| 70 | cyclopropylmethyl | CH₃ | Et | CH₃ | CH₃ |
| 71 | 3-pyridylmethyl | CH₃ | Et | CH₃ | CH₃ |
| 72 | 2-methyl-4-CF₂H-5- | CH₃ | Et | CH₃ | CH₃ |
| | thiazolylmethyl | | | | |
| 73 | 1-methyl-3-CF₃-5-OCF₂H-4- | CH₃ | Et | CH₃ | CH₃ |
| | pyrazolylmethyl | | | | |
| 74 | 1-methyl-3-CF₃-5- | CH₃ | Et | CH₃ | CH₃ |
| | OCH₂Si(CH₃)₃-4- | | | | |
| | pyrazolylmethyl | | | | |
| 75 | 1-methyl-3-CF₂H-4- | CH₃ | Et | CH₃ | CH₃ |
| | pyrazolylmethyl | | | | |
| 76 | 2-methyl-4-CF₃-5- | CH₃ | Et | CH₃ | CH₃ |
| | thiazolylmethyl | | | | |
| 77 | 2-thienyl-2-ethyl | CH₃ | Et | CH₃ | CH₃ |
| 78 | 2-Cl-4,5 methylenedioxy- | CH₃ | Et | CH₃ | CH₃ |
| | benzyl | | | | |
| 79 | 3-Si(CH₃)₃-propyl | CH₃ | Et | CH₃ | CH₃ |
| 80 | n-hexyl | CH₃ | Et | Br | CH₃ |
| 81 | cyclohexylmethyl | CH₃ | Et | Br | CH₃ |
| 82 | 2-oxo-2-phenylethyl | CH₃ | Et | CH₃ | CH₃ |
| 83 | 3-CH(OCH₃)₂-(2-oxo-1,3- | CH₃ | Et | CH₃ | CH₃ |
| | oxazolydin-5-yl)-methyl | | | | |

Table 3 indicates the results of the GC-MS analyses on compounds 5-83.

TABLE 3

| Compound | GC-MS: |
|---|---|
| Nr. | M+ |
| 5. | 358 |
| 6. | 432 |
| 7. | 353 |
| 8. | 466 |
| 9. | 367 |
| 10. | 323 |
| 11. | 279 |
| 12. | 338 |
| 13 | 386 |
| 14 | 448 |
| 15 | 331 |
| 16. | 313 |
| 17. | 307 |
| 18. | 415 |
| 19. | 331 |
| 20. | 320 |
| 21. | 335 |
| 22. | 363 |
| 23. | 363 |
| 24. | 372 |
| 25. | 389 |
| 26. | 413 |
| 27. | 360 |
| 28. | 337 |
| 29. | 378 |
| 30. | 334 |
| 31. | 335 |
| 32. | 360 |
| 33. | 339 |
| 34. | 339 |
| 35. | 320 |
| 36. | 363 |
| 37. | 327 |
| 38. | 291 |
| 39. | 350 |
| 40 | 320 |
| 41 | 453 |
| 42. | 424 |
| 43 | 400 |
| 44. | 498 |
| 45. | 370 |
| 46. | 372 |
| 47 | 468 |
| 48 | 332 |
| 49 | 400 |
| 50. | 328 |
| 51. | 440 |
| 52. | 307 |
| 53. | 328 |
| 54. | 382 |
| 55. | 400 |
| 56. | 348 |
| 57. | 393 |
| 58. | 334 |
| 59. | 334 |
| 60. | 459 |
| 61. | 461 |
| 62. | 498 |
| 63. | 306 |
| 64. | 292 |
| 65. | 484 |
| 66. | 315 |
| 67. | 315 |
| 68. | 321 |
| 69. | 322 |
| 70. | 278 |
| 71. | 315 |
| 72. | 385 |
| 73. | 452 |
| 74. | 488 |
| 75. | 377 |
| 76. | 403 |
| 77. | 334 |
| 78. | 392 |
| 79. | 338 |
| 80. | 373 |
| 81. | 385 |
| 82. | 360 |
| 83. | 397 |

### Example 12

Determination of the preventive fungicidal activity (5 days) against Puccinia Recondita on wheat.

Leaves of wheat plants of the Salgemma variety, grown in pots in a conditioned environment at 20° C. and 70% of Relative Humidity (RH) were treated by spraying both sides of the leaves with the compound under examination (see Table 4 hereunder) dispersed in a hydroacetonic solution at 20% by volume of acetone.

After remaining 5 days in a conditioned environment, the plants were sprayed on both sides of the leaves with an aqueous suspension of conidia of Puccinia Recondita (2 mg of inoculum per 1 ml of solution for infection).

After spraying, the plants were kept in a humidity-saturated environment at a temperature ranging from 18 to 24° C. for the incubation period of the fungus (1 day).

At the end of this period, the plants were put in a greenhouse with a relative humidity (RH) of 70% and at a temperature of 18-24° C. for 14 days.

At the end of this period, the external symptoms of the pathogen appeared and it was therefore possible to proceed with the visual evaluation of the intensity of the infection, both on the parts treated directly with the products (T) and on the parts developed during the implementation of the test (NT).

The fungicidal activity is expressed as a percentage of the reduction, with respect to non-treated seedlings (comparison), in the area of the leaf affected by the disease (100=full effectiveness; 0=zero effectiveness).

All of the compounds 1, 2, 3 and 5 showed full activity (100%) at the dosage of 250 ppm.

At the same time, an evaluation of the phytotoxicity was effected (percentage of leaf necrosis) induced on the wheat seedlings by the application of the products: in this case the evaluation scale ranges from 0 (completely healthy plant) to 100 (completely necrotic plant).

Table 4 indicates the results obtained by carrying out the test described on compounds 1, 2, 3, 5, 17, 39 and 40 compared with a compound described in WO2012/146125:

CR1: N-ethyl-N-methyl-N′-[5-bromo-2-methyl-6-(3-methyl-butyloxy)-3-pyridyl]-formamidine (compound nr. Q.391 of WO′125).

TABLE 4

| Compound | | Activity P5 | | Phytotoxicity |
|---|---|---|---|---|
| Nr. | ppm | T | NT | % N.F. |
| 1 | 125 | 98 | 75 | 0 |
| | 250 | 100 | 85 | 0 |
| 2 | 125 | 100 | 85 | 0 |
| | 250 | 100 | 95 | 0 |
| 3 | 125 | 100 | 93 | 0 |
| | 250 | 100 | 98 | 0 |
| 5 | 125 | 85 | 45 | 0 |
| | 250 | 100 | 65 | 0 |
| 17 | 125 | 95 | 55 | 0 |
| | 250 | 98 | 65 | 0 |
| 39 | 125 | 95 | 88 | 0 |
| | 250 | 98 | 97 | 0 |
| 40 | 125 | 80 | 55 | 0 |
| | 250 | 90 | 70 | 0 |
| CR1 | 125 | 60 | 20 | 5 |
| | 250 | 80 | 50 | 10 |

As can be seen from the table, compounds 1, 2, 3, 5, 17, 39 and 40 are effective in containing the disease and do not show any symptoms of phytotoxicity on the plant, unlike the reference compound CR1.

It should also be pointed out that compound 5, a direct analogue of the compound CR1, differentiating only in the substitution of the oxygen atom with a sulfur atom, proves to be more active also at low dosages (125 ppm).

### Example 13

Determination of the preventive fungicidal activity (5 days) against Uromyces Appendiculatus on beans.

Bean plants cv. Borlotto of Vigevano, grown in pots in a conditioned environment, were treated by spraying both sides with the products under examination in a hydroacetonic solution with 20% by volume of acetone (vol./vol.).

After remaining 5 days in a conditioned environment at 23° C. and 70% of relative humidity, the plants were sprayed on the lower side with an aqueous suspension of spores of Uromyces Appendiculatus (200,000 spores/cc); after remaining 24 hours in a humidity-saturated environment, they were re-transferred to the conditioned environment.

After this period, the external symptoms of the pathogen appeared and it was therefore possible to proceed with the visual evaluation of the intensity of the infection.

The fungicidal activity is expressed as a percentage of the reduction, with respect to non-treated seedlings (comparison), in the area of the leaf affected by the disease (100=full effectiveness; 0=zero effectiveness).

All of the compounds 1, 2, 3 showed full effectiveness (100%) at the dosage of 125 ppm.

At the same time, an evaluation of the phytotoxicity was effected (percentage of leaf necrosis) induced on the bean seedlings by the application of the products: in this case the evaluation scale ranges from 0 (completely healthy plant) to 100 (completely necrotic plant).

Table 5 indicates the results obtained by carrying out the test described with compounds 1, 2, 3, 17, 39 and 40, compared with a compound described in WO2012/146125:

CR2: N-ethyl-N-methyl-N′-[5-bromo-2-methyl-6-[(4-methylpentyloxy]pyridyl-3-formamidine (compound nr. P26 of WO′125).

TABLE 5

| | | Activity | Phytotoxicity |
|---|---|---|---|
| Compound | ppm | P5 | % N.F. |
| 1 | 30 | 90 | 0 |
| | 125 | 100 | 0 |
| 2 | 30 | 100 | 0 |
| | 125 | 100 | 0 |
| 3 | 30 | 100 | 0 |
| | 125 | 100 | 0 |
| 17 | 30 | 100 | 0 |
| | 125 | 100 | 0 |
| 39 | 30 | 100 | 0 |
| | 125 | 100 | 0 |
| 40 | 30 | 100 | 0 |
| | 125 | 100 | 0 |
| CR2 | 30 | 80 | 2 |
| | 125 | 90 | 5 |

The compounds according to the present invention tested showed an optimum effectiveness also at very low dosages (30 ppm), without any symptoms of phytotoxicity.

### Example 14

Determination of the preventive fungicidal activity (7 days) against Sphaerotheca Fuliginea on cucumbers.

Cucumber plants cv. Lungo of China, grown in pots in a conditioned environment were treated by spraying both sides with the products under examination in a hydroacetonic solution with 20% by volume of acetone (vol./vol.).

7 days after the treatment, the plants were sprayed on the upper side with an aqueous suspension of spores of Sphaerotheca Fuliginea (200,000 spores/cc); they were then re-transferred to the conditioned environment.

At the end of the period of incubation (8 days), the evaluation of the intensity of the infection was finally effected.

The fungicidal activity is expressed as a percentage of the reduction, with respect to non-treated seedlings (comparison), in the area of the leaf affected by the disease (100=full effectiveness; 0=zero effectiveness).

All of the compounds 1, 2, 3 showed full effectiveness (100%) at the dosage of 125 ppm.

At the same time, an evaluation of the phytotoxicity was effected (percentage of leaf necrosis) induced on the cucumber seedlings by the application of the products: in this case the evaluation scale ranges from 0 (completely healthy plant) to 100 (completely necrotic plant).

Table 6 indicates the results obtained by carrying out the test described with compounds 1, 2, 3 compared with a compound described in WO2012/146125:

CR2: N-ethyl-N-methyl-N′-[5-bromo-2-methyl-6-[(4-methyl pentyloxy]pyridyl-3-formamidine (compound nr. P26).

TABLE 6

| | | Activity | Phytotoxicity |
|---|---|---|---|
| Compound | ppm | P5 | % N.F. |
| 1 | 30 | 90 | 0 |
| | 125 | 100 | 0 |
| 2 | 30 | 99 | 0 |
| | 125 | 100 | 2 |
| 3 | 30 | 98 | 1 |
| | 125 | 100 | 3 |
| CR2 | 30 | 75 | 2 |
| | 125 | 85 | 5 |

The compounds according to the present invention tested, showed an optimum effectiveness also at very low dosages (30 ppm), contrary to the reference compound CR2.

### Example 15

Determination of the preventive activity (7 days) of the compounds having formula (I) against Parastagonospora nodorum on wheat.

Soft wheat plants, of the Abate variety, grown in pots having a diameter of 15 cm, in a conditioned environment (20±1° C. and 70% of Relative Humidity—RH) having reached the appropriate development stage (7 weeks after sowing), were treated by spraying both sides of the leaves with the products under examination. 7 days after treatment, the plants were inoculated with an aqueous suspension of spores of Parastagonospora nodorum (1,000,000 spores/cc+Tween 20-1 drop/100 ml) by spraying both sides of the leaves, using a compressed air gun.

After remaining 48 hours in a humidity-saturated environment, at 21° C., the plants were transferred for the incubation period (10-12 days) to a conditioned environment at 70% of R.H. and at a temperature of 24° C.

At the end of this period, the external symptoms of the pathogen appeared and it was therefore possible to proceed with the visual evaluation of the intensity of the infection.

The fungicidal activity was expressed as a percentage of the reduction, with respect to non-treated seedlings (comparison), in the area of the leaf affected by the disease (100=full effectiveness; 0=zero effectiveness). Table 7 indicates the results obtained by carrying out the test described with compound Nr. 12, compared with a compound described in WO2008/101682:

CR3: N-ethyl-N-methyl-N′-[5-bromo-2-methyl-6-[(1-methyl -2-propoxyethoxy]pyridyl-3-formamidine (compound nr. A1.355).

TABLE 7

| Compound | | Activity |
|---|---|---|
| Nr. | ppm | P7 |
| 12 | 250 | 90 |
| CR3 | 250 | 57 |

Compound 12, according to the present invention, showed an optimum effectiveness on Parastagonospora nodorum contrary to the compound of the known art CR3.

### Example 16

Determination of the preventive fungicidal activity (7 days) against Helmintosporium teres on barley.

Leaves of barley plants (cultivar Gemini), grown in pots in a conditioned environment (20±1° C. and 70% of Relative Humidity—RH) were treated by spraying both sides of the leaves with the compounds under examination, dispersed in a hydroacetonic solution with 20% by volume of acetone.

After remaining 7 days in a conditioned environment, the plants were sprayed on both sides of the leaves with an aqueous suspension of conidia of Helmintosporium teres (50,000 conidia/cc+Tween 20-1 drop/100 ml).

The plants were then kept in a controlled environment during the incubation period of the fungus (wet room 1 day for infection, 3 days in a cell with 70% of R.H. and at a temperature of 0° C. for the incubation period, 3 days for completion in a wet room, biological cycle 12 days).

At the end of this period (12 days), the fungicidal activity was evaluated according to an evaluation percentage scale from 0 (completely infected plant) to 100 (healthy plant).

Table 8 shows the results obtained by carrying out the test described with compound Nr. 8, compared with a compound described in WO2008/101682:

CR4: N-ethyl-N-methyl-N′-[5-bromo-2-methyl-6-[(4-chloro-3-trifluoromethyl]phenoxy]pyridyl-3-formamidine (compound Nr. P29).

TABLE 8

| Compound | | Activity |
|---|---|---|
| Nr. | ppm | P7 |
| 8 | 125 | 30 |
| CR4 | 125 | 0 |

Compound 8, according to the present invention, showed a higher effectiveness with respect to the reference compound CR4, completely inactive on this pathogen.

### Example 17

Stability tests of the compositions containing compounds having formula (I) in a solvent

In order to evaluate the chemical stability of the compounds having Formula (I) in a solvent, 5% solutions were prepared, containing 250 mg of compounds Nr. 1, 17, 39 and 40, dissolved in 4.75 g of various commercial solvents used for the preparation of emulsifiable concentrates.

The resulting solutions were put in an oven at 54° C. for 2 weeks (according to Cipac MT 46.1 update 2012) and then analyzed by means of HPLC in order to verify the concentration of the active ingredient contained therein. The percentage of degradation of the compound having Formula (I) was therefore obtained, after the accelerated stability test.

The relative degradation percentages are indicated in Table 9.

TABLE 9

| | Compound | Compound | Compound | Compound |
|---|---|---|---|---|
| Solvent | Nr. 1 | Nr. 17 | Nr 39 | Nr. 40 |
| ADMA 10 | 1.07% | 1.57% | 0% | 1.09% |
| FMPC | 1.46% | 0.79% | 1.53% | 0% |
| Propylene | 3% | 0% | 0.61% | 0% |
| carbonate | | | | |
| Purasolv BL | 100% | 100% | 100% | 100% |
| RPDE | 0.78% | 1.93% | 1.13% | 1.71% |
| Green 25 | 2.07% | 2.96% | 1.84% | 2.82% |
| CME | 10.79% | 16% | 17.31% | 12.76% |
| Alkamuls T/20 | 58.61% | 74.78% | 79.78% | 81.02% |

ADMA 10=:N,N-dimethyldecanamide;

FMPC=mixture of N-formylmorpholine and propylene carbonate;

RDPE=mixture of dimethyl glutarate, dimethyl succinate and dimethyl adipate;

Green25=mixture of dimethylsulfoxide, acetophenone, 2-ethylhexyl acetate;

Purasolv BL=butyl lactate;

CME=rapeseed methyl ester;

Alkamuls T/20=ethoxylated sorbitan monolaurate.

As can be seen, the emulsifiable concentrates according to the present invention, i.e. prepared with the compounds having formula (I) and with the solvents ADMA 10, FMPC, propylene carbonate, RPDE and Green 25, allow the chemical stability of the compound having Formula (I) to be maintained.

Other solvents used in this field, such as Purasolv BL, CME, Alkamuls T/20, on the contrary, are not suitable for the preparation of emulsifiable concentrates with the compounds having formula (I) according to the present invention, as the emulsifiable concentrates of compounds having formula (I) are not stable in these solvents.

Please amend the claims pursuant to the provisions of 37 C.F.R. § 1.121 as follows:

1. Pyridyl-formamidines having general formula (I): 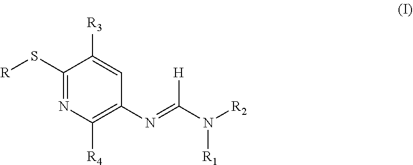
wherein:
R represents a hydrogen; a C₁-C₁₂ alkyl; a C₁-C₁₂ haloalkyl; a C₂-C₁₂ alkenyl; a C₂-C₁₂ haloalkenyl; a C₂-C₁₂ alkinyl; a C₂-C₁₂ haloalkinyl; a C₃-C₁₄ cycloalkyl; a C₄-C₁₈ cycloalkylalkyl; a C₃-C₁₄ cycloalkenyl; a C₃-C₁₄ halocycloalkyl; a C₄-C₁₈ cycloalkenylalkyl; a formyl; a C₂-C₁₂ alkylcarbonyl; a C₂-C₁₂ haloalkylcarbonyl; a C₃-C₁₂ alkenylcarbonyl; a C₄-C₁₄ cycloalkylcarbonyl; or
R represents a C₁-C₆-alkyl-B—C₁-C₁₂-alkyl; C₁-C₆-haloalkyl-B—C₁-C₁₂-alkyl; C₁-C₆-alkyl-B—C₁-C₁₂-haloalkyl; C₁-C₆-haloalkyl-B—C₁-C₁₂-haloalkyl; C₃-C₈-C₃-C₁₄-cyclo-alkyl-B—C₁-C₁₂-alkyl; C₃-C₁₄-cyclo alkyl-B—C₁-C₁₂-halo-alkyl; C₁-C₆-alkyl-B—C₃-C₁₄-cyclo alkyl; C₁-C₆-alkyl-B—C₃-C₁₄-halocycloalkyl; C₃-C₁₄-cyclo-alkyl-B—C₃-C₁₄-cyclo-alkyl; C₄-C₁₈-cycloalkylalkyl-B—C₃-C₁₄-cycloalkyl; C₁-C₆-alkyl-B—C₂-C₁₂-alkenyl; or
R represents A-; A-(C₁-C₆ alkyl)-; A-(C₁-C₆ haloalkyl)-; A-(C₃-C₁₄ cycloalkyl)-; A-(C═O)—; A-(C₁-C₆ alkyl)-(C═O)—; A-B—(C₁-C₁₂ alkyl)-; A-B—(C₁-C₁₂ haloalkyl); A-B—(C₃-C₁₄ cycloalkyl)-; A-(C₁-C₁₂ alkyl)-B—(C₁-C₁₂ alkyl); A-(C₁-C₁₂ alkyl)-B—(C₁-C₁₂-haloalkyl) ; A-B-A-; (C₁-C₆ alkyl)-B-A-; (C₁-C₆ haloalkyl)-B-A-; (C₃-C₁₄ cycloalkyl)-B-A-; A-B-A-(C₁-C₆ alkyl)-; A-B-A-(C₃-C₁₄ cycloalkyl)-; (C₁-C₆ alkyl)-B-A-(C₁-C₆ alkyl)-; (C₃-C₁₄ cycloalkyl)-B-A-(C₁-C₆ alkyl)-; (C₁-C₆ haloalkyl)-B-A-(C₁-C₆ alkyl)-;
A represents an aromatic mono- or bicyclic carbocyclic group possibly substituted by one or more groups, the same or different, preferably selected from the group consisting of halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl; or a condensed monocycle or bicycle with 3-12 terminals, possibly aromatic, partially or completely saturated and which contains from 1 to 4 heteroatoms selected from the group consisting of nitrogen, oxygen and sulfur, with the proviso that these cyclic systems with 3-12 terminals do not contain —O—O—, —S—S—, —O—S— fragments, said cyclic systems with 3-12 terminals being possibly substituted by one or more groups, the same or different, preferably selected from the group consisting of halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ halo alkoxyl groups, a cyano group, a hydroxyl;
B represents —(C═O)—; —C(═NOR₅)—; O—(C═O)—; —C(═O)—O—; —O—; —S—; —N(R₆)—(C═O)—; or —(C═O)—N(R₆)—;
R₁ represents a C₁-C₆ alkyl,
R₂ represents a C₂-C₆ alkyl;
or R₁ and R_{2,} jointly with the N atom to which they are bound, form a heterocyclic ring containing from 4 to 7 atoms, possibly substituted by halogen atoms;
R₃ and R_{4,} the same or different, represent a hydrogen atom; a halogen atom; a C₁-C₆ alkyl; a C₁-C₆ alkoxyl; a C₁-C₆ haloalkoxyl, a CF₃ group; a CF₂H group; a CFH₂ group; a cyano group;
R₅ and R₆ represent a hydrogen atom; a C₁-C₆ alkyl, a C₁-C₆ haloalkyl; a C₃-C₆ cycloalkyl; a benzyl or aryl group possibly substituted by one or more groups, the same or different, preferably selected from the group consisting of halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl;
with the proviso that when R₃ is a hydrogen atom, a halogen atom, a cyano group, a C₁-C₆ alkyl or a C₁-C₆ alkoxyl, R₄ is different from a hydrogen atom, a halogen atom or a cyano group.

2. The pyridyl-formamidines according to claim 1, wherein:
R represents a C₁-C₁₂ alkyl, a C₁-C₁₂ haloalkyl, a C₂-C₁₂ haloalkenyl, a C₃-C₁₄ cycloalkyl, a C₄-C₁₈ cycloalkylalkyl, A-, A-(C₁-C₆ alkyl);
A represents an aromatic mono- or bicyclic carbocyclic group possibly substituted by one or more groups, the same or different, preferably selected from the group consisting of halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ haloalkoxyl groups, a cyano group, a hydroxyl; or a condensed monocycle or bicycle with 3-12 terminals, possibly aromatic, partially or completely saturated and which contains from 1 to 4 heteroatoms selected from nitrogen, oxygen and sulfur, with the proviso that these cyclic systems with 3-12 terminals do not contain —O—O—, —S—S—, —O—S—fragments, said cyclic systems with 3-12 terminals being possibly substituted by one or more groups, the same or different, preferably selected from the group consisting of halogen atoms, C₁-C₁₂ alkyl groups, C₁-C₁₂ haloalkyl groups, C₁-C₆ alkoxyl groups, C₄-C₁₅ cycloalkoxyl groups, C₁-C₆ halo alkoxyl groups, a cyano group, a hydroxyl;
R₁ represents a C₁-C₆ alkyl;
R₂ represents a C₂-C₆ alkyl;
R₃ and R₄ represent a halogen atom, a C₁-C₆ alkyl, with the proviso that when R₃ is a halogen atom, R₄ is not a halogen atom.

3. The pyridyl-formamidines according to claim 1, wherein R₃ and R₄ represent a C₁-C₆ alkyl or R, R₁, R₂, R₃ and R₄ have the meanings indicated in Table 1: 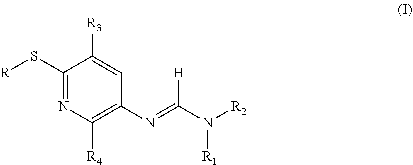
TABLE 1

| Compound | | | | | |
|---|---|---|---|---|---|
| Nr. | R | R1 | R2 | R3 | R4 |
| 1. | 3-CF₃-benzyl | CH₃ | Et | Br | CH₃ |
| 2. | 3-methyl-1-butyl | CH₃ | Et | CH₃ | CH₃ |
| 3. | 3-CF₃-benzyl | CH₃ | Et | CH₃ | CH₃ |
| 4. | 3-CH₃-benzyl | CH₃ | Et | CH₃ | CH₃ |
| 5. | 3-methyl-1-butyl | CH₃ | Et | Br | CH₃ |
| 6. | 3-CF₃-phenyl | CH₃ | Et | Br | CH₃ |
| 7. | 3-CF₃-phenyl | CH₃ | Et | H | CH₃ |
| 8. | 4-Cl-3-CF₃-phenyl | CH₃ | Et | Br | CH₃ |
| 9. | 3-CF₃-phenyl | CH₃ | Et | CH₃ | CH₃ |
| 10. | 3-propoxy-2-propyl | CH₃ | Et | Br | CH₃ |
| 11. | 3-methyl-1-butyl | CH₃ | Et | H | CH₃ |
| 12. | 3-propoxy-2-propyl | CH₃ | Et | CH₃ | CH₃ |
| 13. | 5-CH₃-1,3,4-thiadiazol-2-yl | CH₃ | Et | Br | CH₃ |
| 14. | 3-CF₃-phenyl | CH₃ | Et | Br | OMe |
| 15. | 3,4,4-trifluorobut-3-en-1-yl | CH₃ | Et | CH₃ | CH₃ |
| 16. | benzyl | CH₃ | Et | CH₃ | CH₃ |
| 17. | n-hexyl | CH₃ | Et | CH₃ | CH₃ |
| 18. | 4-Cl-3-CF₃_benzyl | CH₃ | Et | CH₃ | CH₃ |
| 19. | 2-fluoro-benzyl | CH₃ | Et | CH₃ | CH₃ |
| 20. | 3-methyl-thiazol-2-yl | CH₃ | Et | CH₃ | CH₃ |
| 21. | 2-ethyl-1-hexyl | CH₃ | Et | CH₃ | CH₃ |
| 22. | 3-CF₂H-benzyl | CH₃ | Et | CH₃ | CH₃ |
| 23. | n-decyl | CH₃ | Et | CH₃ | CH₃ |
| 24. | 1-adamantyl | CH₃ | Et | CH₃ | CH₃ |
| 25. | cyclododecyl | CH₃ | Et | Br | CH₃ |
| 26. | 5-Br-2-CH₃-thiazol-4-yl | CH₃ | Et | CH₃ | CH₃ |
| 27. | (3-tertbutyl-5-isoxazolyl)methyl | CH₃ | Et | CH₃ | CH₃ |
| 28. | 2-isopropoxy-carbonyl-1-ethyl | CH₃ | Et | CH₃ | CH₃ |
| 29. | 5,5-dimethyl-3-methoxyimino-1-pentyl | CH₃ | Et | Br | CH₃ |
| 30. | 2-(cyclopropyl-aminocarbonyl)-1-ethyl | CH₃ | Et | CH₃ | CH₃ |
| 31. | 4,4-dimethyl-3-oxa-1-pentyl | CH₃ | Et | Br | CH₃ |
| 32. | (2,2-dichloro-1-methyl-cyclopropyl)-methyl | CH₃ | Et | CH₃ | CH₃ |
| 33. | 3-(ethylthio)-1-butyl | CH₃ | Et | CH₃ | CH₃ |
| 34. | 3-[3-(2-oxa-propyl)-cyclohexyl]-1-propyl | CH₃ | Et | CH₃ | CH₃ |
| 35. | 5,5-dimethyl-3-isoxazolyl | CH₃ | Et | Br | CH₃ |
| 36. | (2-naphthyl)methyl | CH₃ | Et | CH₃ | CH₃ |
| 37. | 2-phenyl-1-ethyl | CH₃ | Et | CH₃ | CH₃ |
| 38. | cyclopropanoyl | CH₃ | Et | CH₃ | CH₃ |
| 39. | 2,6-difluorobenzyl | CH₃ | Et | CH₃ | CH₃ |
| 40. | Cyclohexylmethyl | CH₃ | Et | CH₃ | CH₃ |
| 41 | 3-CF₃-phenyl | CH₃ | Et | Br | Cl |
| 42 | 3-methyl-1-butyl | CH₃ | Et | Br | Br |
| 43 | 2-fluoro-3-CF₃-benzyl | CH₃ | Et | CH₃ | CH₃ |
| 44 | 3-CF₃-phenyl | CH₃ | Et | Br | Br |
| 45 | 4-t-butyl-benzyl | CH₃ | Et | CH₃ | CH₃ |
| 46 | 1-adamantyl-methyl | CH₃ | Et | CH₃ | CH₃ |
| 47 | 2-fluoro-4,6 diCF₃-benzyl | CH₃ | Et | CH₃ | CH₃ |
| 48 | 4-fluoro-benzyl | CH₃ | Et | CH₃ | CH₃ |
| 49 | 4-F-3-CF₃-benzyl | CH₃ | Et | CH₃ | CH₃ |
| 50 | 1-phenyl-ethyl | CH₃ | Et | CH₃ | CH₃ |
| 51 | 2-iodo-benzyl | CH₃ | Et | CH₃ | CH₃ |
| 52 | cyclohexyl | CH₃ | Et | CH₃ | CH₃ |
| 53 | 2-CH₃-benzyl | CH₃ | Et | CH₃ | CH₃ |
| 54 | 2-CF₃-benzyl | CH₃ | Et | CH₃ | CH₃ |
| 55 | 2-fluoro-6-CF₃-benzyl | CH₃ | Et | CH₃ | CH₃ |
| 56 | 2-chloro-benzyl | CH₃ | Et | CH₃ | CH₃ |
| 57 | 2-bromo-benzyl | CH₃ | Et | CH₃ | CH₃ |
| 58 | 2-(cyclohexyl)-ethyl | CH₃ | Et | CH₃ | CH₃ |
| 59 | cyclooctyl | CH₃ | Et | CH₃ | CH₃ |
| 60 | 2-Br-6-OCF₂H-benzyl | CH₃ | Et | CH₃ | CH₃ |
| 61 | 2-Br-6-CF₃-benzyl | CH₃ | Et | CH₃ | CH₃ |
| 62 | 2-OCF₂CF₂H-6-CF₃-benzyl | CH₃ | Et | CH₃ | CH₃ |
| 63 | Cyclopentylmethyl | CH₃ | Et | CH₃ | CH₃ |
| 64 | Cyclopentyl | CH₃ | Et | CH₃ | CH₃ |
| 65 | 1-methyl-3-CF₃-5-OCH₂CF₃-4-pyrazolylmethyl | CH₃ | Et | CH₃ | CH₃ |
| 66 | 4-pyridylmethyl | CH₃ | Et | CH₃ | CH₃ |
| 67 | 2-pyridylmethyl | CH₃ | Et | CH₃ | CH₃ |
| 68 | 5,5 dimethyl-2 isoxazolin-3-yl | CH₃ | Et | CH₃ | CH₃ |
| 69 | 5-CH₃-1,3,4-thiadiazol-2-yl | CH₃ | Et | CH₃ | CH₃ |
| 70 | cyclopropylmethyl | CH₃ | Et | CH₃ | CH₃ |
| 71 | 3-pyridylmethyl | CH₃ | Et | CH₃ | CH₃ |
| 72 | 2-methyl-4-CF₂H-5-thiazolylmethyl | CH₃ | Et | CH₃ | CH₃ |
| 73 | 1-methyl-3-CF₃-5-OCF₂H-4-pyrazolylmethyl | CH₃ | Et | CH₃ | CH₃ |
| 74 | 1-methyl-3-CF₃-5-OCH₂Si(CH₃)₃-4-pyrazolylmethyl | CH₃ | Et | CH₃ | CH₃ |
| 75 | 1-methyl-3-CF₂H-4-pyrazolylmethyl | CH₃ | Et | CH₃ | CH₃ |
| 76 | 2-methyl-4-CF₃-5-thiazolylmethyl | CH₃ | Et | CH₃ | CH₃ |
| 77 | 2-thienyl-2-ethyl | CH₃ | Et | CH₃ | CH₃ |
| 78 | 2-Cl-4,5 methylenedioxy-benzyl | CH₃ | Et | CH₃ | CH₃ |
| 79 | 3-Si(CH₃)₃-propyl | CH₃ | Et | CH₃ | CH₃ |
| 80 | n-hexyl | CH₃ | Et | Br | CH₃ |
| 81 | cyclohexylmethyl | CH₃ | Et | Br | CH₃ |
| 82 | 2-oxo-2-phenylethyl | CH₃ | Et | CH₃ | CH₃ |
| 83 | 3-CH(OCH₃)₂-(2-oxo-1,3-oxazolidin-5-yl)-methyl | CH₃ | Et | CH₃ | CH₃ |

4. The pyridyl-formamidines according to claim 1, wherein R, R₁, R₂, R₃ and R₄ have the following meanings:

| Compound | | | | | |
|---|---|---|---|---|---|
| Nr. | R | R₁ | R₂ | R₃ | R₄ |
| 1. | 3-CF₃-benzyl | CH₃ | Et | Br | CH₃ |
| 2. | 3-methylbutyl | CH₃ | Et | CH₃ | CH₃ |
| 3. | 3-CF₃-benzyl | CH₃ | Et | CH₃ | CH₃ |
| 17. | n-hexyl | CH₃ | Et | CH₃ | CH₃ |
| 39. | 2,6-difluorobenzyl | CH₃ | Et | CH₃ | CH₃ |
| 40. | cyclohexylmethyl | CH₃ | Et | CH₃ | CH₃ |

5. The pyridyl-formamidines according to claim 1, which are a) mixtures of non-separated geometric isomers, mixtures of partially separated geometric isomers, single geometric isomers; b) in the form of salts obtained by the addition of inorganic or organic acids.

6. A fungicidal composition comprising one or more compounds having formula (I) according to claim 1, a solvent and/or solid or liquid diluent, and optionally a surfactant.

7. The fungicidal composition according to claim 6, wherein said composition is formulated as an emulsifiable concentrate based on propylenecarbonate, N,N-dimethyloctanamide, N,N-dimethyldecanamide, acetophenone 2-ethythexyl acetate, alkyl esters of adipic acid, alkyl esters of glutaric acid, alkyl esters of succinic; acid, dimethyl sulfoxide or based on morpholine solvents, preferabl N-formylmorpholine, alone or mixed with each other, in a quantity ranging from 2% to 60% by weight with respect to the total weight of the fungicidal composition.

8. The fungicidal composition according to claim 6, further comprising one or more surfactants selected from the group consisting of sodium or calcium or potassium alkylarylsulfonates and combinations thereof, preferably calcium dodecylbenzenesulfonate, or polyethoxylated or polypropoxy-polyethoxylated arylphenols, preferably ethoxylated-propoxylated polyarylphenols and combinations thereof.

9. The fungicidal composition according to claim 6, comprising at least one compound having general formula (I) and at least another active ingredient compatible therewith, selected from the group consisting of fungicides different from those having general formula (I), phytoregulators, antibiotics, herbicides, insecticides, fertilizers, biostimulants and/or combinations thereof, preferably a fungicide belonging to the following classes:
a) azoles selected from the group consisting of azaconazole, bitertanol, bromuconazole, cyproconazole, difenoconazole, epoxyconazole, fenbuconazole, fluquinconazole, flusilazole, flutriafol, hexaconazole, imazalil, ipconazole, metconazole, myclobutanil, penconazole, propiconazole, prochloraz, prothioconazole, sime-conazole tebuconazole, tetraconazole, triadimefon, triadimenol, triflumizole, triticonazole and combinations thereof;
b) amines, ergosterol biosynthesis inhibitors selected from the group consisting of aldimorph, dodemorph, fenpropimorph, fenpropidin, spiroxamine, tridemorph and combinations thereof;
c) succinate-dehydrogenase inhibitors (SDHI) selected from the group consisting of benzovindiflupyr, bixafen, boscalid, carboxin, fluindapyr, fluopyram, flutolanil, fluxapyroxad, furametpyr, isopyrazam, oxycarboxin, penflufen, penthiopyrad, sedaxane, thifluzamide and combinations thereof;
d) strobilurins selected from the group consisting of azoxystrobin, dimoxystrobin, fluoxastrobin, kresoxim-methyl, metominostrobin, orysastrobin, picoxystrobin, pyraclostrobin, pyrametostrobin, pyraoxostrobin, trifloxystrobin and combinations thereof;
e) specific antioidic compounds selected from the group consisting of cyflufenamid, flutianil, metrafenone, proquinazid, pyriofenone, quinoxyfen and combinations thereof;
f) aniline-pyramidines selected from the group consisting of pyrimethanil, mepanipyrim, cyprodini and combinations thereof 1;
g) benzimidazoles and analogues thereof selected from the group consisting of carbendazim, benomyl, thiabendazole, thiophanate-methyl and combinations thereof;
h) dicarboxyimides selected from the group consisting of iprodione, procymidone and combinations thereof;
i) phtalimides selected from the group consisting of captafol, captan, folpet and combinations thereof;
l) systemic acquired resistance (SAR) inducers selected from the group consisting of acibenzolar, probenazole, isotianil, tiadinil and combinations thereof;
m) phenylpyrroles selected from the group consisting of fenpiclonil, fludioxonil and combinations thereof;
n) acylalanines selected from the group consisting of benalaxyl, benalaxyl-M, furalaxyl, metalaxyl, metalaxyl-M and combinations thereof;
o) other specific antiperonosporic compounds selected from the group consisting of ametoctradin, amisulbrom, benthiavalicarb, cyazofamid, cymoxanil, dimethomorph, ethaboxam, famoxadone, fenamidone, flumetover, flumorph, fluopicolide, iprovalicarb, mandipropamid, oxathiapiproline, vali-fenalate and combinations thereof;
p) dithiocarbamates selected from the group consisting of maneb, mancozeb, propineb, zineb and combinations thereof;
q) phosphorous acid and its inorganic and organic salts, fosetyl-aluminium and combinations thereof;
r) rameic compounds selected from the group consisting of Bordeaux mixture, carpropamid, copper hydroxide, copper oxychloride, copper sulfate, copper salycilate and combinations thereof;
s) other fungicides selected from the group consisting of chlorothalonil, fenhexamid, fenpyrazamine, fluazinam, sylthiofam, tebufloquin, zoxamide, dodine, guazatine, iminoctadine and combinations thereof.

10. The fungicidal compound according to claims 6, comprising at least one compound having general formula (I) and at least another known fungicide selected from the group consisting of the following compositions C₁-C₈₁ and combinations thereof:
C₁: compound 3+tetraconazole;
C_{2:} compound 3+tebuconazole;
C_{3:} compound 3+epoxyconazole;
C_{4:} compound 3+prothioconazole;
C_{5:} compound 3+prochloraz;
C_{6:} compound 3+fenpropimorph;
C_{7:} compound 3+spiroxamine;
C_{8:} compound 3+bixafen;
C_{9:} compound 3+boscalid;
C_{10:} compound 3+carboxin;
C_{11:} compound 3+fluopyram;
C_{12:} compound 3+fluxapyroxad;
C_{13:} compound 3+isopyrazam;
C_{14:} compound 3+penthiopyrad;
C_{15:} compound 3+sedaxane;
C_{16:} compound 3+azoxystrobin;
C_{17:} compound 3+dimoxystrobin;
C_{18:} compound 3+fluoxastrobin;
C_{19:} compound 3+kresoxim-methyl;
C_{20:} compound 3+picoxystrobin;
C_{21:} compound 3+pyraclostrobin;
C_{22:} compound 3+trifloxystrobin;
C_{23:} compound 3+metrafenone;
C_{24:} compound 3+proquinazid;
C_{25:} compound 3+mepanipyrim;
C_{26:} compound 3+cyprodinil;
C_{27:} compound 3+iprodione;
C_{28:} compound 3+procymidone;
C_{29:} compound 3+carbendazim;
C_{30:} compound 3+thiophanate-methyl;
C_{31:} compound 3+3 fluindapyr;
C_{32:} compound 3+benalaxyl-M;
C_{33:} compound 3+benzovindiflupyr;
C_{34:} compound 1+tetraconazole;
C_{35:} compound 1+fluindapyr;
C_{36:} compound 1+azoxystrobin;
C_{37:} compound 1+pyraclostrobin;
C_{38:} compound 2+tetraconazole;
C_{39:} compound 2+tebuconazole;
C_{40:} compound 2+epoxyconazole;
C_{41:} compound 2+prothioconazole;
C_{42:} compound 2+prochloraz;
C_{43:} compound 2+fenpropimorph;
C_{44:} compound 2+spiroxamine;
C_{45:} compound 2+bixafen;
C_{46:} compound 2+boscalid;
C_{47:} compound 2+carboxin;
C_{48:} compound 2+fluopyram;
C_{49:} compound 2+fluxapyroxad;
C_{50:} compound 2+isopyrazam;
C_{51:} compound 2+penthiopyrad;
C_{52:} compound 2+sedaxane;
C_{53:} compound 2+azoxystrobin;
C_{54:} compound 2+dimoxystrobin;
C_{55:} compound 2+fluoxastrobin;
C_{56:} compound 2+kresoxim-methyl;
C_{57:} compound 2+picoxystrobin;
C_{58:} compound 2+pyraclostrobin;
C_{59:} compound 2+trifloxystrobin;
C_{60:} compound 2+metrafenone;
C_{61:} compound 2+proquinazid;
C_{62:} compound 2+mepanipyrim;
C_{63:} compound 2+cyprodinil;
C_{64:} compound 2+iprodione;
C_{65:} compound 2+procymidone;
C_{66:} compound 2+carbendazim;
C_{67:} compound 2+thiophanate-methyl;
C_{68:} compound 2+fluindapyr;
C_{69:} compound 2+benalaxyl-M;
C_{70:} compound 2+benzovindiflupyr;
C_{71:} compound 2+tetraconazole+azoxystrobin,
C_{72:} compound 2+pyraclostrobin+tetraconazole;
C_{73:} compound 2+epoxyconazole+azoxystrobin;
C_{74:} compound 2+pyraclostrobin+epoxyconazole;
C_{75:} compound 3+azoxystrobin+fluindapyr;
C_{76:} compound 3+pyraclostrobin+fluindapyr;
C_{77:} compound 3+fluindapyr;
C_{78:} compound 3+tetraconazole+azoxystrobin;
C_{79:} compound 3+pyraclostrobin+tetraconazole;
C_{80:} compound 3+azoxystrobin+fluindapyr;
C_{81:} compound 3+fluindapyr+tetraconazole.

11. Use of compounds having formula (I) according to claims 1 for the control of phytopathogenic fungi of agricultural crops, of both a curative and preventive nature.

12. Use according to claim 11, for the control of phytopathogenic fungi of agricultural crops wherein the phytopathogenic fungi belong to the following classes: Basidiomycetes, Ascomycetes, Deuteromycetes or imperfect fungi, Oomycetes, preferably Puccinia spp., Ustilago spp., Tilletia spp., Uromyces spp., Phakopsora spp., Rhizoctonia spp., Erysiphe spp., Sphaerotheca spp., Podosphaera spp., Uncinula spp., Helminthosporium spp., Rhynchosporium spp., Pyrenophora spp., Monilinia spp., Sclerotinia spp., Septoria spp. (Mycosphaerella spp.), Venturia spp., Botrytis spp., Alternaria spp., Fusarium spp., Cercospora spp., Cercosporella herpotrichoides, Colletotrichum spp., Pyricularia oryzae, Sclerotium spp., Phytophtora spp., Pythium spp., Plasmopara viticola, Peronospora spp., Pseudoperonospora cubensis, Bremia lactucae, and/or wherein the agricultural crops are selected from cereals, such as wheat, barley, rye, oats, rice, corn, sorghum; fruit-trees such as apples, pears, plums, peaches, almonds, cherries, bananas, grapes, strawberries, raspberries, blackberries; citrus fruits such as oranges, lemons mandarins, grapefruit; legumes such as beans, peas, lentils, soybeans; vegetables such as spinach, lettuce, asparagus, cabbage, carrots, onions, tomatoes, potatoes, eggplants, peppers; cucurbits such as pumpkins, zucchini, cucumbers, melons, watermelons; oleaginous plants such as sunflowers, rapeseed, peanuts, castor, coconuts; tobacco, coffee, tea, cocoa, sugar beet, sugar cane, cotton.

13. Use according to claim 11, for the control of Plasmopara viticola on vines, Phytophtora infestans and Botrytis Cinerea on tomatoes, Puccinia Recondita, Erisiphae Graminis, Helminthosporium Teres, Parastagonospora nodorum, Zymoseptoria Tritici and Fusarium spp. on cereals, Phakopsora Pachyrhizion soybeans, Uromyces Appendiculatus on beans, Venturia Inaequalis on apples, Sphaerotheca Fuliginea on cucumbers.

14. Use of compounds having formula (I) according to claims 1 for the control of phytopathogenic bacteria and viruses, preferably Xanthomonas spp., Pseudomonas spp., Erwinia Amylovora, the tobacco mosaic virus.

15. A method for controlling phytopathogenic fungi in agricultural crops, which consists of applying effective and non-phytotoxic doses of compounds having formula (I) according to claims 1.

16. Use of compounds having fungicidal compositions according to claim 6 for the control of phytopathogenic fungi of agricultural crops, of both a curative and preventive nature.

17. Use of compounds having fungicidal compositions according to claim 6 for the control of phytopathogenic bacteria and viruses, preferably Xanthomonas spp., Pseudomonas spp., Erwinia Amylovora, the tobacco mosaic virus.

18. A method for controlling phytopathogenic fungi in agricultural crops, which consists of applying effective and non-phytotoxic doses of fungicidal compositions according to claim 6.
